(19) United States
(12) Patent Application Publication
(10) Pub. No.: US 2022/0117555 A1
(43) Pub. Date: Apr. 21, 2022

(54) SYSTEM FOR MONITORING OR TREATING A MEDICAL CONDITION OF A PATIENT

(71) Applicant: VentriMend, Inc., Lake Forest, CA (US)

(72) Inventors: David Zarbatany, Laguna Niguel, CA (US); Randell Werneth, San Diego, CA (US); Jeffrey Castleberry, Longmont,, CO (US); Ricardo Roman, Chula Vista, CA (US); R. Maxwell Flaherty, Topsfield, MA (US); J. Christopher Flaherty, Auburndale, FL (US)

(73) Assignee: VentriMend, Inc., Lake Forest, CA (US)

(21) Appl. No.: 17/427,880

(22) PCT Filed: Jan. 4, 2019
(86) PCT No.: PCT/US19/12304

§ 371 Date: Aug. 2, 2021

Related U.S. Application Data
(60) Provisional application No. 62/614,372, filed on Jan. 6, 2018.

(51) Int. Cl. A61B 5/00; A61B 5/363; A61B 5/0215; A61B 5/024; A61B 5/145; A61B 5/1468

(52) U.S. Cl. CPC A61B 5/686; A61B 5/6869; A61B 5/6851; A61B 5/6882; A61B 5/6862; A61B 5/363; A61B 5/0006; A61B 5/0036; A61B 5/024; A61B 5/14539; A61B 5/14542; A61B 5/1468; A61B 5/0215

(57) ABSTRACT

A system for treating and/or monitoring a medical condition of a patient comprises: an implant configured to be inserted into a body space of the patient, such as the left atrial appendage. The implant comprises a frame or ring configured to engage tissue of the patient proximate the body space. Methods of treating and/or monitoring a medical condition of a patient are also provided.

### RELATED APPLICATIONS

The present application is a national stage application under 35 U.S.C. § 371 of PCT Application No. PCT/US2019/012304, filed Jan. 4, 2019, which claims benefit of priority to U.S. Provisional Application No. 62/614,372, titled “System For Monitoring Or Treating A Medical Condition Of A Patient”, filed Jan. 6, 2018, and is related to U.S. patent application Ser. No. 13/368,685, titled “Atrial Appendage Occlusion and Arrhythmia Treatment”, filed Feb. 8, 2012, the content of each of which are incorporated herein by reference in their entirety for all purposes.

### INCORPORATION BY REFERENCE

All publications and patent applications mentioned in this specification are herein incorporated by reference to the same extent as if each individual publication or patent application was specifically and individually indicated to be incorporated by reference.

### FIELD OF THE INVENTION

The present invention relates generally to medical systems including cardiac implants, particularly to systems that include at least one implant positioned in the left atrial appendage.

### BACKGROUND

The left atrial appendage (“LAA”) is a cavity extending from the lateral wall of the left atrium between the mitral valve and the root of the left pulmonary veins. The LAA normally contracts with the rest of the left atrium during a normal heart cycle, keeping blood from becoming stagnant therein, but often fails to contract with any vigor in patients experiencing atrial fibrillation (“AF”) due to the discoordinate electrical signals associated with AF, in patients with AF and other abnormal heart conduction. The result is that blood tends to pool in the LAA, which can lead to the formation of blood clots therein. The blood clots can then propagate out from the LAA into the left atrium. Since blood from the left atrium and ventricle supply the heart and brain, blood clots from the LAA can obstruct blood flow thereto, causing heart attacks, strokes, or other organ ischemia. Blood clots form in the LAA in about 90% of patients with atrial thrombus. Patients with AF account for one of every six stroke patients, and thromboemboli originating from the LAA are the suspected culprit in the vast majority of these cases. More than 3 million Americans have AF, which increases their risk of stroke by a factor of 5. Elimination or containment of thrombus formed within the LAA of patients with AF will significantly reduce the incidence of stroke in those patients.

LAA occlusion can be used as an alternative for patients who cannot use oral anticoagulants such as warfarin. Approximately 17% of patients cannot take anticoagulants because of a recent or previous bleeding, non-compliance, or pregnancy. Current US FDA-approved occlusion methods staple the LAA closed or suture and excise the appendage. Studies, however, have shown these techniques produce inconsistent results. Some new approaches, currently under FDA investigation, deliver an implant from within the vascular system.

The left atrial appendage (LAA) has been identified as an important source of AF triggers, particularly among patients with structural heart disease, nonparoxysmal AF, and AF recurrence after AF ablation. LAA electrical isolation (LAAEI) has been viable adjunctive strategy to treating patients with AF in addition to PV isolation. LAAEI is an adjunctive strategy to PV isolation for maintenance of SR. Mechanical force displaced radially at the ostium of the LAA. Will create electrical isolation by compressing the myocyte cells at the contact site and inhibit the exchange of sodium and calcium thus elimination of the refractory process of cardiac myocytes. The resulting cellular response causes apoptosis or programmed cellular death, this process decouples active cells causing electrically deactivated cells. This produces a focal line of non-conductive tissue, ultimately causing tissue necrosis electrically disassociating the LA from LAA tissue.

In AF, the LAA can cause a significant amount of arrhythmogenic sources (ectopic activity, PV-like potentials) which is an important initiating source of AF, in patients with previous ablation procedures, the LAA can continue to initiate and/or maintain the AF arrhythmia. Left atrial appendage electrical isolation in addition to standard ablation will have an incremental benefit to achieve freedom from ALL atrial arrhythmias in patients with atrial fibrillation. The appendage has limited contractibility when in AF and if it is isolated it would have no contractibility, combining LAA electrical isolation with a LAA polymer filling the LAA would ensure patient safety and improve AF outcomes while reducing stroke.

Percutaneous LAA occlusion has been demonstrated to be as effective as anticoagulation drugs in reducing the risk of thromboembolic stroke in patients with AF. The combination of a standard AF ablation procedure with LAA electrical isolation and LAA occlusion will be an elegant method of improving success rates of ablation for AF whilst also mitigating stroke risk and reducing the bleeding risks from long-term anticoagulation.

Devices are needed, however, to more consistently and effectively prevent clots from forming and/or entering the atrium from the left atrial appendage.

### SUMMARY

Embodiments of the systems, devices and methods described herein can be directed to systems, devices and methods for treating and/or diagnosing a cardiac condition.

According to an aspect of the present inventive concepts, a system for treating and/or monitoring a medical condition of a patient, comprises: an implant configured to be inserted into a body space of the patient, and the implant comprises a frame or ring configured to engage tissue of the patient proximate the body space.

In some embodiments, the body space comprises the left atrial appendage (LAA).

In some embodiments, the system is configured to monitor and/or treat a cardiac condition selected from the group consisting of: atrial fibrillation (AF); atrial flutter; ventricular tachycardia; supraventricular tachycardias; bradycardia; congestive heart failure; mitral valve insufficiency; tricuspid valve insufficiency; ischemia; and combinations thereof.

In some embodiments, the system is configured to record a patient parameter selected from the group consisting of: blood pressure; blood pressure proximate the heart; ECG; and combinations thereof.

In some embodiments, the system is configured to stimulate the heart of the patient, such as to pace the heart, defibrillate the heart, and/or deliver a cardiac drug to the patient.

In some embodiments, the system is configured to electrically isolate the LAA from the heart. The implant can be configured to impart a force to electrically isolate the LAA. The implant can be configured to deliver ablative energy to electrically isolate the LAA.

In some embodiments, the system is configured to perform two or more of: electrically isolate the LAA; fluidly isolate the LAA; record one or more patient physiologic parameters of the patient; and/or stimulate the heart of the patient.

In some embodiments, the implant further comprises a covering (also referred to as a “cover”) that surrounds at least a portion of the frame or ring. The covering can comprise a material selected from the group consisting of: a woven material; a fabric; a wire mesh; polyethylene terephthalate; a sponge; cellulose; synthetic fiber; cotton, rayon, hydrogel; a coagulant; a biodegradable material; a non-biodegradable material, and combinations thereof.

In some embodiments, the implant further comprises at least one anchor. The anchor can comprise a material configured to absorb fluids.

In some embodiments, the implant further comprises at least one sensor. The at least one sensor can comprise one, two, or more sensors selected from the group consisting of: an electrode; an electrical activity sensor; a heart rate sensor; a pressure sensor; a pH sensor; a blood sensor; a blood gas sensor; a chemical sensor; and combinations of one, two, or more of these. The at least one sensor can be configured to record: ECG; left atrial pressure; heart rate; and combinations of one, two or more of these. The at least one sensor can be configured to measure the pressure at a location selected from the group consisting of: within the LA; within a pulmonary vein; and combinations thereof.

In some embodiments, the implant further comprises an electronics module. The electronics module can be configured to: collect and/or process information; store information; transmit information; receive energy; and/or transmit energy. The implant can further comprise at least one sensor, and the electronics module can comprise an algorithm configured to analyze data recorded by the at least one sensor.

In some embodiments, the frame comprises a ring like portion configured to engage tissue. The ring like portion can be configured to engage the ostium of the left atrium. The ring portion can comprise an adjustable diameter.

In some embodiments, the implant further comprises fill material. The fill material can be configured to absorb blood and/or other fluids. The fill material can be biodegradable, non-biodegradable, or a combination of biodegradable and non-biodegradable. The fill material can comprise multiple fill portions. The fill material can comprise a geometry customized based on the patient's anatomy. The implant can be configured to fluidly isolate the left atrial appendage. The implant can be further configured to electrically isolate the left atrial appendage. The implant can be further configured to fluidly isolate and electrically isolate at the same time. The fill material can comprise at least one of radiopaque material or a radiopaque marker, identifier.

In some embodiments, the implant comprises a first implant that includes a sensor that records electrical activity, and the system further comprises a second implant comprising a stimulation device, and the second implant is configured to pace the patient's heart when the system detects an abnormal cardiac rhythm based on the recorded electrical activity.

According to an aspect of the present inventive concepts, a system for treating, monitoring, analyzing and communicating a medical condition of a patient, comprises: an implant configured to be inserted into or near the left atrial appendage (LAA).

In some embodiments, the implant is adapted to prevent clots from entering the left atrium from the left atrial appendage.

In some embodiments, the implant is configured to electrically isolate the LAA from the heart. The implant can be configured to impart a force to electrically isolate the LAA. The implant can be configured to deliver ablative energy to electrically isolate the LAA. The implant can be configured to impart a force and deliver ablative energy at the same time to electrically isolate the LAA

In some embodiments, the implant has a component placed at the ostium of the LAA, this “cover” prevents blood from entering the LAA and from contacting the implanted polymer and creates electrical isolation through the pressure applied to the ostial tissue by the outer ring. The cover comprises a ring portion configured to engage LAA ostial tissue. The ring portion can be configured to engage the ostium of the left atrium. The ring portion can comprise an adjustable diameter and shape.

In some embodiments, the implant comprises a first implant that includes a sensor that records left atrial pressure, and the system further comprises a second implant comprising an implant that includes a sensor that records electrical activity, and in another configuration a stimulation device, and the next implant is configured to pace the patient's heart when the system detects an abnormal cardiac rhythm based on the recorded electrical activity.

In some embodiments, the system is configured to record and communicate externally a patient parameter selected from the group consisting of: blood pressure; blood pressure proximate the heart; blood velocity, ECG; and combinations thereof.

In some embodiments, the system is configured to stimulate the heart of the patient, such as to pace the heart, defibrillate the heart, and/or deliver a cardiac drug to the patient.

The technology described herein, along with the attributes and attendant advantages thereof, will best be appreciated and understood in view of the following detailed description taken in conjunction with the accompanying drawings in which representative embodiments are described by way of example.

### BRIEF DESCRIPTION OF THE DRAWINGS

FIG. 1 illustrates a schematic view of a medical system including a cardiac implant, consistent with the present inventive concepts.

FIG. 2 illustrates an anatomical sectional view of the delivery of a cardiac implant, consistent with the present inventive concepts.

FIG. 3 illustrates an anatomical sectional view of a device implanted in the left atrial appendage, consistent with the present inventive concepts.

FIGS. 4A-B illustrate a side view of two embodiments of an anchor for a cardiac implant, consistent with the present inventive concepts.

FIG. 5 illustrates an anatomical sectional view of an implant positioned in the left atrial appendage, consistent with the present inventive concepts.

FIG. 6 illustrates an anatomical sectional view of another implant positioned in the left atrial appendage, consistent with the present inventive concepts.

FIGS. 7A-C illustrate anatomical sectional views of a series of steps of implanting a device that electrically and fluidly isolates the left atrial appendage, consistent with the present inventive concepts.

FIGS. 8A-C illustrate anatomical sectional views of a series of steps of implanting a device that fluidly isolates the left atrial appendage, consistent with the present inventive concepts.

FIG. 9 illustrates an anatomical sectional view of a system configured to fluidly isolate the Left Atrial Appendage (“LAA”) from the left atrium (“LA”).

FIGS. 10A-10C illustrate anatomical sectional views of a series of steps of implanting a device that fluidly isolates the left atrial appendage.

FIG. 10D illustrates a perspective view of a split isolation ring having features of the invention.

FIGS. 11A-11B illustrate sectional views of one embodiment of a LAA isolation ring having an outer band with a raised isolation portion or isolation tab configured to apply maximum pressure to the LAA.

FIGS. 11C-11D illustrate perspective views of another embodiment of a LAA isolation ring having an inner ring and an outer ring with two diameters configured to apply maximum pressure to the LAA.

FIG. 11E illustrates a perspective view of another embodiment of a LAA isolation ring having an outer ring for ablation.

FIGS. 12A-12B illustrate perspective views of an Isolation Ring with Left-Atrial Appendage Cover.

FIG. 13 illustrates an anatomical sectional view of an electrical isolation ring with biocompatible material-embedded carbon nanotubes configured to redirect the LAA signal.

FIGS. 14A-14C illustrate sectional views of one embodiment of a LAA ring that provides the combination of pressure mediated ablation plus electrical energy ablation.

FIG. 15A illustrates a perspective view of an Isolation Ring or ostium ring having a plurality of spokes surrounded by an outer ring.

FIGS. 15B-15D illustrate perspective views of alternate types of spokes for the isolation ring.

FIG. 16A illustrates an anatomical sectional view of an implant having a one-way valve configured to allow blood flow into the LAA, but not allow blood flow out of the LAA.

FIG. 16B illustrates an anatomical sectional view of an implant having a two-way valve with a filter or screen that is configured to allow blood flow in and out of the LAA while at the same time the screen prevents clots from coming out of the LAA.

FIGS. 17A-17C illustrate anatomical sectional views of an implant having a cover that includes a self-expanding, disc-like structure, to close the LAA at the ostium's circumference and provide a smooth surface for endothelialization of the implant cover over the LAA ostium.

FIGS. 18A-18C illustrate sectional views of one embodiment of a multi-functional ring/cover configured to provide electrical isolation and mechanical isolation, independently.

FIG. 19 illustrates an anatomical sectional view of a stent ring coupled with a basket type anchor engaging the LAA.

FIGS. 20A-20B illustrate anatomical sectional views of a stent ring coupled with one or more spline anchors engaging the LAA.

FIG. 21A illustrates a front view, and FIGS. 21B and 21C illustrate side views of the stent ring comprising one or more sinusoidal rings and a braided mesh filter cover with filler access.

FIG. 22 illustrates one embodiment of a system having an implant with one or more sensors configured to continuously monitor physiological data (e.g. ECG signals) and/or wirelessly transmit physiological data to an external system or device.

FIG. 23 illustrates one embodiment of a delivery device configured to deliver the fill material to the LAA in a dry condition.

FIG. 24 illustrates another embodiment of fill material twisted like a rope during delivery to prevent fluid absorption.

FIGS. 25A-25C illustrate anatomical sectional views of a series of steps of implanting fill material into the LAA.

FIGS. 26A-26D illustrate anatomical sectional views of a series of steps of delivering an implant with connected fill material sections into the LAA and a cover to seal the LAA.

FIGS. 27A-27C illustrate another embodiment of an implant delivered to the LAA that includes a cover and fill material, delivered in sections, and a disk at the distal end of the fill material, and a center cord that runs the length of the material sections.

FIGS. 28A-28C illustrate one embodiment of a fill material being made of two materials, a polymer outer foam on an inner foam configuration.

FIG. 29A illustrates one embodiment of fill material is a polymerizing fluid fill material within a balloon inserted into the LAA.

FIGS. 29B-29C illustrate one embodiment of fill material is “coils” inserted into the LAA.

FIGS. 30A-30B illustrate one embodiment of a delivery rod having notches.

FIGS. 31A and 31B illustrate one option for delivery of the implant in a catheter using a polymer delivery component that is similar to a guidewire.

FIG. 32 illustrates one embodiment of a delivery device having a transseptal sheath distal mounted expandable LAA occlusion balloon, which is configured to be inserted into the ostium of the LAA and expanded to seal the ostium.

FIGS. 33A-33C illustrate one embodiment of a delivery device having a transseptal sheath distal mounted expandable LAA balloon configured to expand to seal the ostium, and also configured to deliver expandable fill material into the LAA.

FIGS. 34A-34C illustrate a series of steps that fluidly isolates the left atrial appendage, including stitching the LAA closed.

FIGS. 35A-35E illustrate one embodiment for deployment of a blood expanding fill material in the LAA.

FIGS. 36A and 36B show one embodiment of an implant having a single wire-form/braid with a proximal wire mesh LAA ostial ring and a distal wire mesh disk connected by a connective wire mesh cord.

FIG. 37 shows the polymer fill material wrapped around the center connective wire mesh cord connecting the distal wire mesh disk to the larger, proximal wire mesh.

FIG. 38 shows the device being elongated and then compressed for delivery.

FIGS. 39A-39D show deployment of the compressed device from the end of a catheter or sheath.

FIG. 40 shows one embodiment of a device designed to provide a 3-point position concept.

### DETAILED DESCRIPTION OF THE DRAWINGS

Reference will now be made in detail to the present embodiments of the technology, examples of which are illustrated in the accompanying drawings. Similar reference numbers may be used to refer to similar components. However, the description is not intended to limit the present disclosure to particular embodiments, and it should be construed as including various modifications, equivalents, and/or alternatives of the embodiments described herein.

It will be understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.).

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of one or more of these.

As used herein, the term “proximate”, when used to describe proximity of a first component or location to a second component or location, is to be taken to include one or more locations near to the second component or location, as well as locations in, on and/or within the second component or location. For example, a component positioned proximate an anatomical site (e.g. a target tissue location), shall include components positioned near to the anatomical site, as well as components positioned in, on and/or within the anatomical site.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence.

As used herein, the term “threshold” refers to a maximum level, a minimum level, and/or range of values correlating to a desired or undesired state. In some embodiments, a system parameter is maintained above a minimum threshold, below a maximum threshold. within a threshold range of values and/or outside a threshold range of values, to cause a desired effect (e.g. efficacious therapy) and/or to prevent or otherwise reduce (hereinafter “prevent”) an undesired event (e.g. a device and/or clinical adverse event). In some embodiments, a system parameter is maintained above a first threshold (e.g. above a first temperature threshold to cause a desired therapeutic effect to tissue) and below a second threshold (e.g. below a second temperature threshold to prevent undesired tissue damage). In some embodiments, a threshold value is determined to include a safety margin, such as to account for patient variability, system variability, tolerances, and the like. As used herein, “exceeding a threshold” relates to a parameter going above a maximum threshold, below a minimum threshold, within a range of threshold values and/or outside of a range of threshold values.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

In this specification, unless explicitly stated otherwise, “and” can mean “or,” and “or” can mean “and.” For example, if a feature is described as having A, B, or C, the feature can have A, B, and C, or any combination of A, B, and C. Similarly, if a feature is described as having A, B, and C, the feature can have only one or two of A, B, or C.

The expression “configured (or set) to” used in the present disclosure may be used interchangeably with, for example, the expressions “suitable for”, “having the capacity to”, “designed to”, “adapted to”, “made to” and “capable of” according to a situation. The expression “configured (or set) to” does not mean only “specifically designed to” in hardware. Alternatively, in some situations, the expression “a device configured to” may mean that the device “can” operate together with another device or component.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross sectional area as the cross section of the component being described.

The terms “major axis” and “minor axis” of a component where used herein are the length and diameter, respectively, of the smallest volume hypothetical cylinder which can completely surround the component.

As used herein, the term “functional element” is to be taken to include one or more elements constructed and arranged to perform a function. A functional element can comprise a sensor and/or a transducer. In some embodiments, a functional element is configured to deliver energy and/or otherwise treat tissue (e.g. a functional element configured as a treatment element). Alternatively or additionally, a functional element (e.g. a functional element comprising a sensor) can be configured to record one or more parameters, such as a patient physiologic parameter; a patient anatomical parameter (e.g. a tissue geometry parameter); a patient environment parameter; and/or a system parameter. In some embodiments, a sensor or other functional element is configured to perform a diagnostic function. In some embodiments, a functional element comprises one or more elements constructed and arranged to perform a function selected from the group consisting of: deliver energy; extract energy (e.g. to cool a component); deliver a drug or other agent; manipulate a system component or patient tissue; record or otherwise sense a parameter such as a patient physiologic parameter or a patient anatomical parameter; and combinations of one or more of these. A functional element can comprise a fluid, such as an ablative fluid (as described hereabove) comprising a liquid or gas configured to ablate or otherwise treat tissue. A functional element can comprise a reservoir, such as an expandable balloon configured to receive an ablative fluid. A “functional assembly” can comprise an assembly constructed and arranged to perform a function, such as is described hereabove. In some embodiments, a functional assembly is configured to deliver energy and/or otherwise treat tissue (e.g. a functional assembly configured as a treatment assembly). Alternatively or additionally, a functional assembly can be configured to record one or more parameters, such as a patient physiologic parameter; a patient anatomical parameter; a patient environment parameter; and/or a system parameter. A functional assembly can comprise an expandable assembly. A functional assembly can comprise one or more functional elements.

The term “transducer” where used herein is to be taken to include any component or combination of components that receives energy or any input, and produces an output. For example, a transducer can include an electrode that receives electrical energy, and distributes the electrical energy to tissue (e.g. based on the size of the electrode). In some configurations, a transducer converts an electrical signal into any output, such as light (e.g. a transducer comprising a light emitting diode or light bulb), sound (e.g. a transducer comprising a piezo crystal configured to deliver ultrasound energy), pressure, heat energy, cryogenic energy, chemical energy; mechanical energy (e.g. a transducer comprising a motor or a solenoid), magnetic energy, and/or a different electrical signal (e.g. a Bluetooth or other wireless communication element). Alternatively or additionally, a transducer can convert a physical quantity (e.g. variations in a physical quantity) into an electrical signal. A transducer can include any component that delivers energy and/or an agent to tissue, such as a transducer configured to deliver one or more of: electrical energy to tissue (e.g. a transducer comprising one or more electrodes); light energy to tissue (e.g. a transducer comprising a laser, light emitting diode and/or optical component such as a lens or prism); mechanical energy to tissue (e.g. a transducer comprising a tissue manipulating element); sound energy to tissue (e.g. a transducer comprising a piezo crystal); chemical energy; electromagnetic energy; magnetic energy; and combinations of one or more of these.

As used herein, the term “fluid” can refer to a liquid, gas, gel, or any flowable material, such as a material which can be propelled through a conduit of the present inventive concepts.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Provided herein are systems, devices, and methods for monitoring, treating, diagnosing, and/or prognosing a medical condition. One or more implants, such as a cardiac implant, can be implanted in the patient, such as at a location in or proximate the left atrial appendage (LAA) of the patient. The implant can include one or more sensors that monitor one or more physiologic parameters of the patient, such as blood pressure in the left atrium (LA) of the patient.

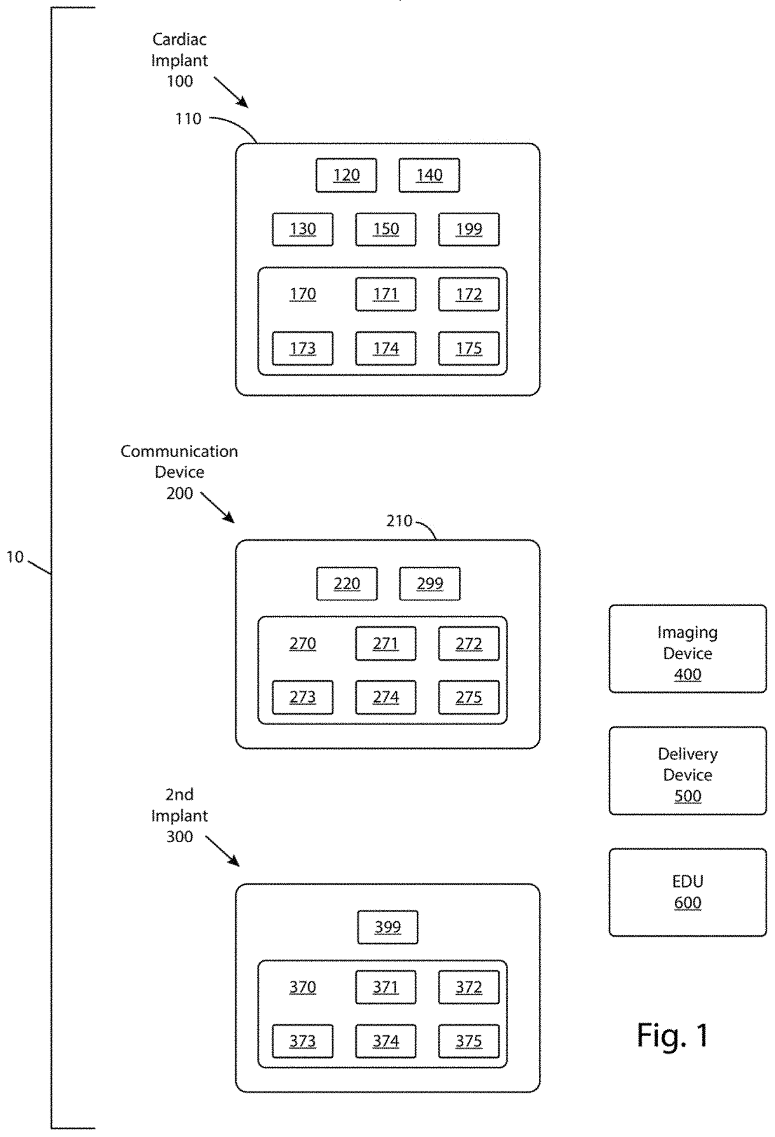

Referring now to FIG. 1, a schematic view of a medical system including a cardiac implant is illustrated, consistent with the present inventive concepts. System 10 includes an implantable device, cardiac implant 100, which can be implanted in the left atrial appendage or other cardiac location. System 10 can further include communication device 200 which is maintained outside of the patient's body, usually in the patient's environment (e.g. home or clinical setting in which the patient resides). Communication device 200 is configured to receive information from cardiac implant 100 and/or transfer information to implant 100. In some embodiments, communication device 200 is configured to transfer energy to implant 100 (e.g. inductive transfer of energy). In some embodiments, system 10 includes another implantable device, second implant 300. In some embodiments, system 10 includes a device for imaging the patient, imaging device 400. In some embodiments, system 10 includes delivery device 500.

System 10 can be configured to monitor, diagnose, prognose, and/or treat one or more patient medical conditions.

In some embodiments, system 10 is configured to monitor and/or treat a cardiac condition, such as: atrial fibrillation (AF); atrial flutter; ventricular tachycardia; supraventricular tachycardia; bradycardia; congestive heart failure; mitral valve insufficiency; tricuspid valve insufficiency; and/or ischemia. For example, implant 100 and/or another component of system 10 can be used to electrically isolate the left atrial appendage (LAA). In some embodiments, implant 100 is further configured to fluidly isolate the LAA (e.g. from the LA).

In some embodiments, system 10 is configured to reduce the risk of embolization, such as to reduce the risk of clot formation and migration within and/or otherwise proximate the LAA, such as when implant 100 is positioned within and/or otherwise proximate the LAA to fluidly isolate the LAA from the left atrium (LA).

In some embodiments, system 10 is configured to record (e.g. continuously and/or intermittently record) one or more patient physiologic parameters, such as blood pressure within and/or at least proximate the heart. For example, blood pressure of the left side of the heart can be monitored to diagnose and/or prognose one or more cardiac diseases. Alternatively or additionally, system 10 can be configured to record electrocardiograms (ECG) of the patient. In some embodiments, system 10 is configured to record parameters selected from the group consisting of: blood pressure; blood flow velocity and/or direction; ECG; and combinations of one, two, or more of these. System 10 can be configured to record these and/or other data for an extended period of time, such as a period of at least 1 week, at least 1 month, or at least 6 months. Data can be recorded continuously and/or intermittently.

In some embodiments, system 10 is configured to stimulate the heart of the patient, such as to pace the heart; defibrillate the heart; and/or deliver a cardiac drug to the patient. In these embodiments, system 10 can be configured to record one or more patient physiologic parameters, and to deliver stimulation based on the recorded parameters (e.g. in a closed loop manner), such as is described herein. System 10 can stimulate the heart through the delivery of energy such as electrical energy and/or via the delivery of a stimulating pharmaceutical or other agent. Stimulation can be delivered by implant 100 and/or second implant 300 (e.g. as delivered by an energy delivery-based and/or agent delivery-based functional element 199 and/or 399, respectively).

In some embodiments, system 10 is configured to perform two or more of: electrically isolate the LAA; fluidly isolate the LAA; record one or more patient physiologic parameters of the patient; and/or stimulate the heart of the patient; each as described herein. In some embodiments, system 10 is configured to perform three or more of: electrically isolate the LAA; fluidly isolate the LAA; record one or more patient physiologic parameters of the patient; and/or stimulate the heart of the patient. In some embodiments, system 10 is configured to perform all four of: electrically isolate the LAA; fluidly isolate the LAA; record one or more patient physiologic parameters of the patient; and stimulate the heart of the patient.

Implant 100 can be implanted in a surgical procedure or via one or more catheter-based delivery devices in an interventional procedure (e.g. an over-the-wire interventional percutaneous procedure). In some embodiments, implant 100 is positioned proximate the LAA of the patient, such as to fluidly isolate the LAA, such as to reduce risk of clot formation and migration that might otherwise result (e.g. in a patient with AF or otherwise compromised LA function). Alternatively or additionally, implant 100 can be positioned proximate the LAA to electrically isolate the LAA (e.g. as a therapeutic treatment for AF). Alternatively or additionally, implant 100 can be positioned proximate the LAA to record one or more physiologic parameters of the patient, such as blood pressure and/or ECG within the LA and/or within the LAA.

Implant 100 can comprise a frame or ring 110, that can be configured to secure implant 100 to heart tissue. Ring 110 can comprise an elastic material, such as a material selected from the group consisting of: nickel titanium alloy; stainless steel; a shaped memory material; a superelastic material; and combinations of one, two, or more of these. In some embodiments, ring 110 comprises a ring-shaped portion that is configured to engage the ostium of the LAA.

Implant 100 can comprise a covering that surrounds at least a portion of ring 110, covering 120. In some embodiments, covering 120 comprises a material selected from the group consisting of: a woven material; a fabric; a wire mesh; polyethylene terephthalate; a sponge; cellulose; synthetic fiber; cotton; rayon; hydrogel; a coagulant; a biodegradable material; a non-biodegradable material and combinations of one, two, or more of these.

Implant 100 can comprise fill material 130, which can be positioned within ring 110. Fill material 130 can comprise a material selected from the group of biocompatibles consisting of: a sponge material; a fabric material; cotton; rayon; a hydrogel; a polymer; and combinations of one, two, or more of these. Alternatively or additionally, fill material 130 can comprise an injectable material, such as an injectable embolization material such as polyvinyl alcohol (PVA). In some embodiments, fill material 130 comprises a material that biodegrades over time, such as a time of at least 1 week, at least 1 month, or at least 6 months, and/or a time of no more than 1 month, no more than 3 months, or no more than 6 months.

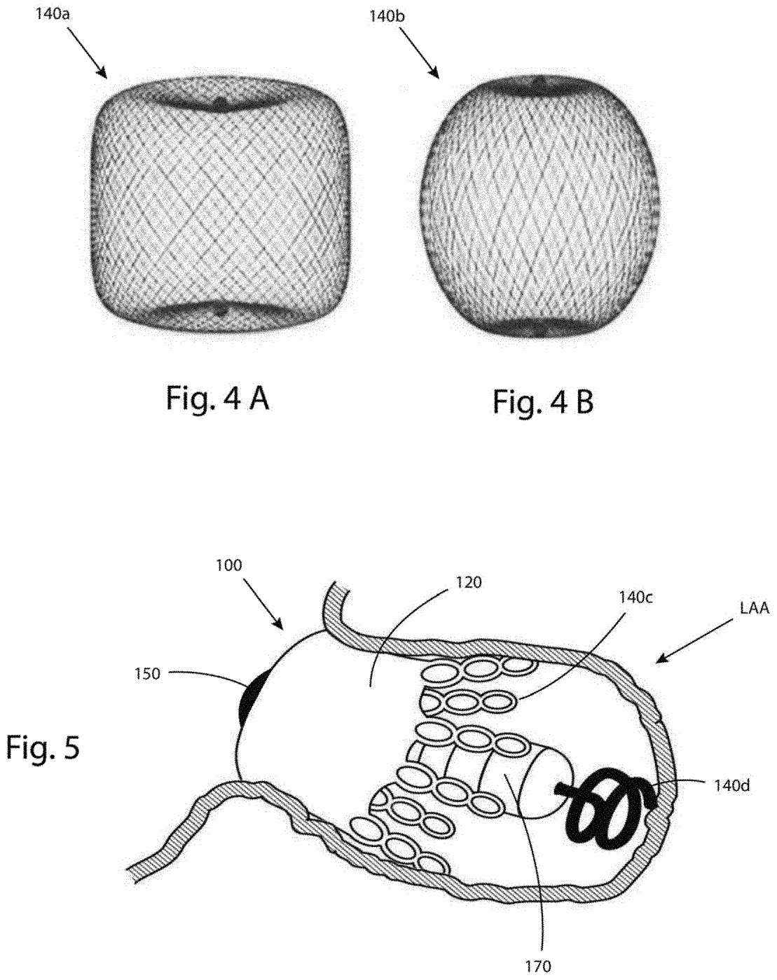

Implant 100 can comprise anchor 140, comprising one or more anchoring elements (e.g. in addition to or as an alternative to ring 110 functioning as an anchoring element). Anchor 140 can comprise one or more coils, such as is described here below in reference to FIG. 3. Alternatively or additionally, anchor 140 can comprise one or more wire meshes, materials which absorb fluids, such as is described here below in reference to FIGS. 4A-B.

Anchor 140 may comprise a wire mesh construction in a cylinder configuration and/or spherical configuration. Anchor 140 can be attached to ring 110 such that anchor 140 is positioned in the LAA. In some embodiments, an embolic agent is positioned within and/or around anchor 140 (e.g. fill material 130 injected during the delivery of anchor). Anchor 140 may comprise single-layer or multilayer construction. Anchor 140 can be configured to provide rapid intraprocedural stasis (e.g. stasis of blood within the LAA).

Anchors 140 may be attached at one or more locations of ring 110. Anchors 140 may comprise materials selected from the group consisting of: nickel titanium alloy; stainless steel; a shaped memory material; a polymer; and combinations of one, two, or more of these.

Implant 100 can include one or more sensors, sensor 150, such as one, two, or more sensors selected from the group consisting of: an electrode; an electrical activity sensor; a heart rate sensor; a pressure sensor; a pH sensor; a blood sensor; a blood gas sensor; a chemical sensor; and combinations of one, two, or more of these. In some embodiments, sensor 150 is configured to record: ECG; left atrial pressure; heart rate; and combinations of one, two or more of these. In some embodiments, fill material 130, covering 120, electronics module 170, sensor 150, and/or ring 110 includes a radiopaque material and/or includes a radiopaque marker.

Implant 100 can comprise electronics module 170, comprising various components configured to collect and/or process information, store information, transmit information, receive energy (e.g. from communication device 200) and/or transmit energy (e.g. to one or more electrodes or other energy delivery components of implant 100 as described herein). Electronics module 170 can comprise an energy storage element, power supply 171 (e.g. a battery, a rechargeable battery, and/or a capacitor). Electronics module 170 can comprise memory circuitry, memory 172, configured to store information. Electronics module 170 can comprise a microcontroller or data processing assembly, processing unit 173. Electronics module 170 can comprise one or more algorithms, algorithm 174, such as an algorithm configured to analyze data recorded by implant 100. Electronics module 170 can comprise a module configured to transmit data (e.g. to a separate device within or outside of the patient), transmitter 175. In some embodiments, transmitter 175 is configured to receive data, such as when transmitter 175 transmits and/or receives data to and/or from communication device 200 and/or second implant 300. Transmitter 175 can comprise one or more antennas configured to transmit and/or receive data and/or power (e.g. power received from communication device 200, and/or power transmitted to second implant 300).

One or more portions of implant 100 can be configured to unfold and/or otherwise expand during and/or after deployment (e.g. delivery into the heart using delivery device 500). In some embodiments, implant 100 is configured to be recaptured during and/or after implantation (e.g. during and/or after deployment from delivery device 500). In these embodiments, implant 100 can be configured to be folded or otherwise radially compressed for recapture.

Communication device 200 can comprise one or more communication devices that transfer information with implant 100 and/or other components of system 10 (e.g. second implant 300 and/or imaging device 400).

Communication device 200 includes housing 210 which comprises one or more housings surrounding various components of communication device 200.

Positioned on housing 210 is user interface 220, comprising one or more user input components and/or user output components configured to allow a user (e.g. the patient and/or a clinician of the patient) to enter or receive information.

Communication device 200 can comprise electronics module 270, comprising various components configured to collect and/or process information, store information, transmit information, and/or transmit energy (e.g. transmit energy inductively to implant 100). Electronics module 270 can comprise an energy storage element, power supply 271 (e.g. a battery, a rechargeable battery, and/or a capacitor). Electronics module 270 can comprise memory circuitry, memory 272, configured to store information. Electronics module 270 can comprise a microcontroller or data processing assembly, processing unit 273. Electronics module 270 can comprise one or more algorithms, algorithm 274, such as an algorithm configured to analyze data recorded by implant 100. Electronics module 270 can comprise a module configured to transmit data and/or energy (e.g. to implant 100 and/or to a separate device within or outside of the patient), transmitter 275. In some embodiments, transmitter 275 is configured to receive data, such as when transmitter 275 transmits and/or receives data to and/or from implant 100 and/or second implant 300. Transmitter 275 can comprise one or more antennas configured to transmit and/or receive data and/or energy (e.g. to and/or from communication device 200 and/or second implant 300).

Second implant 300 can be implanted in a surgical procedure or via one or more catheter-based delivery devices in an interventional percutaneous procedure (e.g. an over-the-wire interventional procedure). In some embodiments, second implant 300 is positioned proximate the heart of the patient, such as when second implant 300 is configured to pace and/or defibrillate the patient's heart.

Second implant 300 can comprise electronics module 370, comprising various components configured to collect and/or process information, store information, transmit information, receive energy (e.g. from communication device 200) and/or transmit energy (e.g. to one or more electrodes or other energy delivery components of second implant 300 as described herein). Electronics module 370 can comprise an energy storage element, power supply 371 (e.g. a battery, a rechargeable battery, and/or a capacitor). Electronics module 170 can comprise memory circuitry, memory 372, configured to store information. Electronics module 370 can comprise a microcontroller or data processing assembly, processing unit 373. Electronics module 370 can comprise one or more algorithms, algorithm 374, such as an algorithm configured to analyze data recorded by second implant 300. Electronics module 370 can comprise a module configured to transmit data (e.g. to a separate device within or outside of the patient), transmitter 375. In some embodiments, transmitter 375 is configured to receive data, such as when transmitter 375 transmits and/or receives data to and/or from communication device 200 and/or implant 100. Transmitter 375 can comprise one or more antennas configured to transmit and/or receive data (e.g. to and/or from implant 100 and/or communication device 100), and/or one or more antennas configured to receive energy (e.g. from communication device 200 and/or implant 100).

As described hereabove, in some embodiments second implant 300 transfers information to and/or from implant 100 (e.g. via transmitters 375 and 175). Alternatively or additionally, energy can be transferred between second implant 300 and implant 100, such as via inductive or other wireless energy transfer.

Imaging device 400 can comprise one or more devices configured to provide an image of the patient, such as an imaging device selected from the group consisting of: MRI; X-Ray; Ct-scan; ultrasound imaging device; OCT imaging device; and combinations of one, two, or more of these.

In some embodiments, one or more images are created of the patient's anatomy into which implant 100 is to be subsequently implanted. In these embodiments, one or more components and/or portions of implant 100 (e.g. ring 110, covering 120, fill material 130, implanted sensor 150, and/or electronics module 170) can be fabricated in a customized manner, such that implant 100 efficiently “fits” the implant location of the particular patient receiving implant 100. For example, one or more components and/or portions of implant 100 can be fabricated using 3D printing technologies based on the images of the patient's anatomy created by imaging device 400 (e.g. one, two or more 2D and/or 3D images created by imaging device 400).

Delivery device 500 can comprise one or more catheters or other tools configured to deliver a separate device, such as to deliver implant 100. In some embodiments, delivery device 500 is configured to deliver energy to implant 100. In some embodiments, delivery device 500 is configured to deliver fill material 130 to implant 100 (e.g. after the other components of implant 100 have been positioned in the LAA).

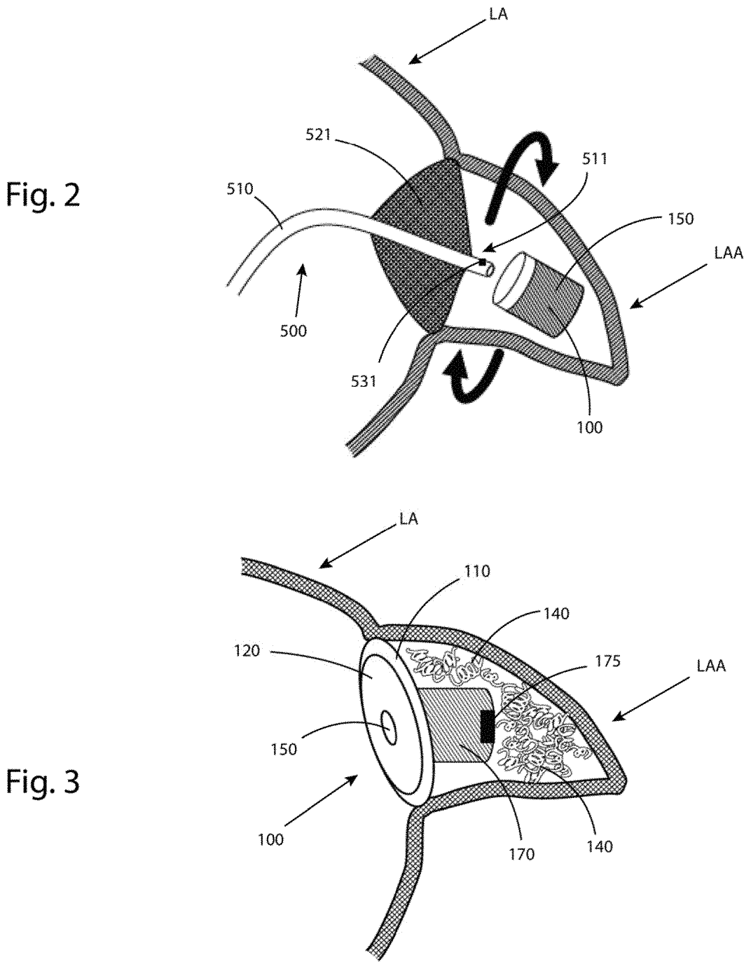

In some embodiments, delivery device 500 is configured to perform a medical procedure, such as is described here below in reference to FIG. 2.

In some embodiments, system 10 comprises one or more functional elements, such as functional elements 199, 299, and 399 shown as integral to implant 100, communication device 200, and/or second implant 300, respectively. Functional elements 199, 299, and/or 399 can each comprise one or more sensors, and/or one or more transducers. In some embodiments, functional elements 199 and/or 399 comprise an electrode or other implantable sensor and/or transducer, such as a sensor configured to record cardiac physiologic information and/or to deliver electrical energy to the heart.

In some embodiments, functional elements 199, 299, and/or 399 comprise a sensor configured to record a physiologic parameter of the patient, such as one or more physiologic parameters selected from the group consisting of: an electrode; an electrical activity sensor; a heart rate sensor; a pressure sensor; a pH sensor; a blood sensor; a blood gas sensor; a chemical sensor; and combinations of one, two, or more of these. In some embodiments, functional elements 199, 299, and/or 399 are configured to record: ECG; left atrial pressure; heart rate; and combinations of one, two or more of these.

In some embodiments, functional elements 199, 299, and/or 399 comprise a transducer configured to deliver energy to pace and/or defibrillate the patient's heart.

In some embodiments, sensor 150 comprises one or more sensors configured to assess one or more physiologic parameters of the patient, such as one or more cardiac physiologic parameters as described herein. In some embodiments, data collected by implant 100 can be processed internally (e.g. via algorithm 174 of implant 100), and/or by a device outside of the patient's body, such as communication device 200. In some embodiments, communication device 200 includes a portion located outside of the patient's environment, such as when communication device 200 includes a portion located at the manufacturer of system 10 that provides a data analysis service. In these embodiments, data from multiple patients can be compared or otherwise analyzed collectively, and/or stored for future analysis.

In some embodiments, sensor 150 comprises one or more sensors configured to determine the pressure within one or more chambers of the heart, such as pressure within the LA when implant 100 is implanted in the LAA, or pressure within a pulmonary vein (e.g. for determining mitral and/or tricuspid valve insufficiency and/or right heart failure). In these embodiments, one or more algorithms of system 10 (e.g. algorithm 174 and/or 274 described herein) can be used to monitor (e.g. diagnose and/or prognose) one or more patient conditions, such as to monitor congestive heart failure of the patient. This monitoring can be used to improve patient care, such as to determine an event selected from the group consisting of: a proper time to discharge the patient from a hospital (e.g. to prevent unnecessary stays in a hospital); a proper time to request the patient be treated in a hospital setting (e.g. to prevent a medical condition from unnecessarily getting worse due to lack of proper care); a proper time to modify the intake of one or more medications (e.g. start a medication; increase a medication; decrease a medication; and/or stop a medication); and combinations of one, two, or more of these.

In some embodiments, sensor 150 comprises a MEMS resonator and/or other MEMS sensor configured to measure pressure (e.g. blood pressure within the LA).

In some embodiments, sensor 150 comprises a sensor of similar construction and arrangement to a sensor configured to be used to measure intraocular pressure.

Ring 110 and/or another portion of implant 100 can be configured to electrically isolate the LAA from the heart. In some embodiments, ring 110 and/or another portion of implant 100 is configured to impart a force (e.g. a force exerted fully circumferentially about the LAA ostium) that results in an electrical isolation of the LAA. At the time of placement of implant 100, the imparted forces disrupt cell to cell conduction within the tissue (e.g. by compressing myocytes to prevent ion exchange). Over time, a biological response occurs (e.g. as a result of the pressure and slight stretching caused by implant 100) that results in endothelial cell formation and fibrous tissue growth in the LAA ostial tissue. The cellular matrix created by implant 100 is non-conductive, providing long term electrical block. In addition, the newly generated tissue can surround ring 110 or other portion of implant 100, such as to prevent movement and migration. In some embodiments, ring 110 and/or another portion of implant 100 is configured to deliver ablative energy to tissue, such as thermal energy (e.g. ablative heat or ablative cooling), radiofrequency (RF) and/or other electrical energy (e.g. ablative electrical energy), and/or sound energy (e.g. ablative ultrasound) energy, such as during delivery when implant 100 is temporarily connected to a source of energy (e.g. electrical, thermal, and/or sound energy provided by an energy delivery unit, EDU 600 shown).

In some embodiments, ring 110 comprises a ring-like portion that engages the ostium of the LAA. The ring-like portion can have an adjustable geometry, such as an adjustable diameter. In some embodiments, the ring 110 is positioned in the LAA ostium, and the diameter of the ring-like portion is increased until electrical isolation of the LAA is achieved (e.g. due to the effect on the tissue described hereabove). The ring-like portion of ring 110 can comprise a shape memory material that changes dimension (increases diameter) in response to an application of energy (e.g. energy delivered using delivery device 500). Alternatively or additionally, the ring-like portion can comprise a ratchet geometry whose diameter can incrementally increase (e.g. via a ratchet-control mechanism of delivery device 500).

One or more portions of implant 100, such as covering 120 and/or fill material 130 can be configured to fluidly isolate the LAA, as described herein. In some embodiments, ring 110 engages the LAA ostium, and covering 120 provides a complete seal between the LA and the LAA, such as is described here below in reference to FIG. 3. Alternatively or additionally, implant 100 can include fill material 130 which can fill the LAA, preventing clot formation that could enter the bloodstream via the LA. Alternatively or additionally, system 10 can be configured to reduce the LAA volume, such as by cinching down the LAA and/or applying a vacuum to the LAA, as described here below in reference to FIG. 2.

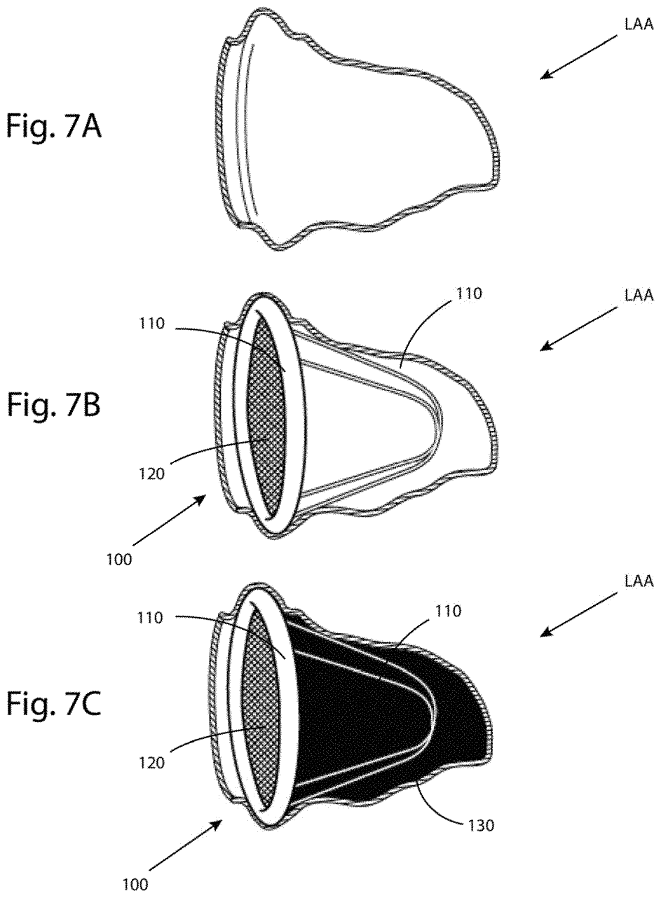

As described herein, in some embodiments, system 10 is configured to both electrically isolate the LAA and provide fluid isolation of the LAA as well. For Example, ring 110 can comprise a ring-like portion that provides electrical isolation of the LAA (e.g. as described herein), and implant 100 can comprise fill material 130 that fluidly isolates the LAA, such as is described here below in reference to FIGS. 7A-C. Fill material 130 can be configured to absorb blood present in the LAA, such absorption causing fill material 130 to expand to approximate the space of the LAA.

As described hereabove, in some embodiments system 10 is configured to stimulate the heart of the patient. In these embodiments, system 10 can be configured to deliver the stimulation in a closed loop fashion, such as based on physiologic parameters recorded by sensor 150 of implant 100 or other sensor of system 10. Sensor 150 can be configured to record ECG of the patient, and second implant 300 can deliver stimulation energy to pace the heart when an abnormal cardiac rhythm is detected, such as when detected by algorithm 174 of implant 100 and/or algorithm 274 of communication device 200. For example, system 10 can be configured to detect atrial fibrillation (AF) and, when detected, cause implant 100 and/or second implant 300 to pace the heart to cardiovert the patient. Alternatively or additionally, system 10 can be configured to detect lack of a pulse, ventricular fibrillation, (VF), and/or pulseless ventricular tachycardia (VT), and, when any are detected, cause implant 100 and/or second implant 300 to defibrillate the heart.

In some embodiments, implant 100 is configured to pace the heart, such as with low energy (e.g. approximately 1 Joule), high frequency (e.g. 0.05 Hz to 40 Hz) pacing energy delivered from within the left atrium, such as to restore sinus rhythm in a closed loop fashion when sensor 150 of implant 100 records electrical activity of the heart that is determined to represent AF as identified by system 100 (e.g. identified via algorithm 174 and/or algorithm 274).

In some embodiments, system 10 is configured to monitor left atrial pressure, as described herein. In these embodiments, system 10 can be used to identify when the patient's left atrial pressure is above a threshold and/or is trending in a direction identifying a deterioration of the patient's health (e.g. peak LA pressure is above a threshold and/or average LA pressure is above a threshold). In these embodiments, a subsequent procedure can be performed to treat the elevated pressure. For example, the patient can be put on a blood pressure reducing medication, a diuretic and/or a blood pressure reducing procedure can be performed such as a fistula created between a conduit of the venous system and a conduit of the arterial system, and/or a shunt between the left atrium and the right atrium. In some embodiments, a shunt is created between the left and right atriums of the heart by inserting a cryoablation balloon through the atrial septum. The balloon can be inflated to a desired diameter (e.g. a balloon at body temperature), after which cryoablation fluid can be delivered to the balloon (e.g. to ablate and/or coagulate).

In some embodiments, implant 100 is configured to reshape a chamber of the heart, such as to reshape the LAA and/or LA. For example, implant 100 can be positioned to reshape tissue proximate the mitral valve (e.g. as a treatment for mitral regurgitation).

Referring now to FIG. 2, an anatomical sectional view of the delivery of a cardiac implant is illustrated, consistent with the present inventive concepts. Delivery device 500 comprises an elongate shaft, shaft 510, with a distal portion 511. Mounted to distal portion 511 is an expandable balloon, balloon 521, which has been inserted into the ostium of the LAA. One or more needles can be included in distal portion 511, needles 531, which can be deployed and subsequently used to cinch down (e.g. via the placement of sutures) the proximal portion of the LAA. Alternatively, delivery device 500 can use clips, adhesive, and/or other fixation means, to cinch down the LAA.

Delivery device 500 can be used to deliver implant 100, prior to and/or after the cinching of the LAA. In these embodiments, one or more portions of implant 100 can be configured to unfold and/or otherwise expand during and/or after deployment from delivery device 500. Implant 100 can comprise sensor 150, such as a pressure sensor and/or an ECG sensor. Implant 100 can be of similar construction and arrangement as implant 100 described hereabove in reference to FIG. 1.

In some embodiments, fill material 130 not shown but described herein, is delivered into the LAA (e.g. using delivery device 500), such as to cause stasis in the LAA. In some embodiments, delivery device 500 can be used to apply a vacuum to an internal portion of the LAA, such as after implant 100 has been secured at the LAA ostium. In these embodiments, the vacuum can be applied to reduce the internal volume of the LAA, which can avoid the need for cinching down the LAA and/or reduce the volume of fill material 130.

Referring now to FIG. 3, an anatomical sectional view of a device implanted in the left atrial appendage is illustrated, consistent with the present inventive concepts. Implantable device 100 has been positioned such that ring 110, positioned on the proximal end of implant 100, engages the ostium of the LAA. Covering 120 covers the area defined by ring 110, and functions as a protective barrier to exclude the LAA as described hereabove (e.g. covering 120 provides a relatively complete seal between the LA and the LAA). Implant 100 can comprise sensors 150 which can be positioned on the surface of covering 120 as shown, or at a location within the LAA. Implant 100 further comprises electronics module 170, which includes transmitter 175. Implant 100 can be of similar construction and arrangement to implant 100 described hereabove in reference to FIG. 1.

Implant 100 of FIG. 3 can include one or more additional anchors (e.g. in addition to ring 110), such as anchor 140 comprising one or more microcoils. Anchor 140 can comprise one or more coils of similar construction and arrangement to treat an aneurysm, such as a brain aneurysm. Anchor 140 can engage tissue of the LAA to anchor implant 100.

In some embodiments, implant 100 includes fill material 130, not shown but such as an injected embolization material or other material configured to fill the LAA around anchors 140. Fill material 130 can comprise an adhesive material. Fill material 130 can be used if covering 120 does not provide a complete seal between the LA and the LAA.

In some embodiments, sensors 150 comprises one or more sensors oriented away from covering 120, such as to measure pressure within the LA while avoiding direct contact with blood circulating in the LA (e.g. reducing the likelihood of a reaction between a sensor of sensor 150 causing thrombus formation and/or a reaction between sensor 150 that deteriorates the performance of the sensor).

Anchors 140 can be attached at one or more locations of ring 110. Ring 110 and/or anchors 140 can comprise materials selected from the group consisting of: nickel titanium alloy; stainless steel; a shaped memory material; a polymer; and combinations of one, two, or more of these.

Referring to FIGS. 4A-B, a side view of two embodiments of an anchor for a cardiac implant are illustrated, consistent with the present inventive concepts. Anchor 140a of FIG. 4A and anchor 140b of FIG. 4B each comprise a wire mesh construction in a cylinder configuration and spherical configuration, respectively. Anchors 140a,b can each be attached to ring 110 of implant 100, such as when attached to a side of ring 110 positioned away from the LA (e.g. such that anchor 140 is positioned in the LAA). In some embodiments, an embolic agent, as described herein, is positioned within and/or around anchor 140 (e.g. fill material 130 injected during the delivery of anchor 140). Anchors 140a,b can comprise single-layer or multilayer construction. Anchors 140a,b can be configured to provide rapid intraprocedural stasis (e.g. stasis of blood within the LAA).

Anchors 140 can be attached at one or more locations of ring 110. Anchors 140 can comprise materials selected from the group consisting of: nickel titanium alloy; stainless steel; a shaped memory material; a polymer; and combinations of one, two, or more of these.

Referring to FIG. 5, an anatomical sectional view of an implant positioned in the left atrial appendage is illustrated, consistent with the present inventive concepts. Implant 100 has been positioned such that a ring-portion of ring 110 (not shown but described herein) is positioned at the ostium of the LAA. Implant 100 includes covering 120 (surrounding ring 110 and providing a barrier between the LA and the LAA), sensor 150 (e.g. a pressure sensor and/or an ECG sensor), and electronics module 170. Implant 100 can comprise one or more anchors 140, such as anchor 140c and/or 140d shown. Implant 100 can be of similar construction and arrangement as implant 100 described hereabove in reference to FIG. 1.

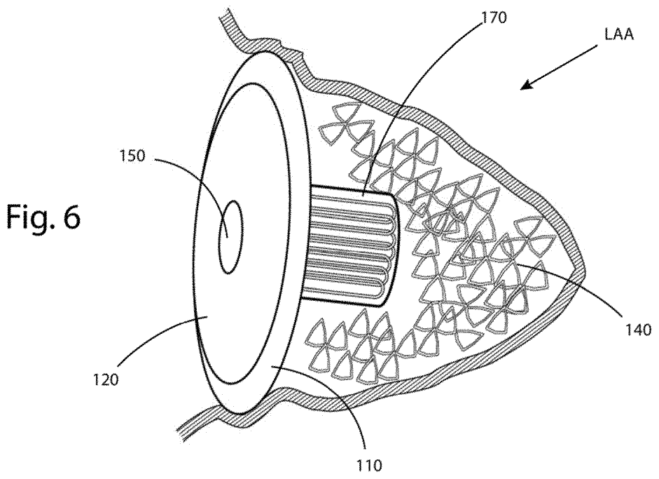

Referring now to FIG. 6, an anatomical sectional view of an implant positioned in the left atrial appendage is illustrated, consistent with the present inventive concepts. Implant 100 includes ring 110 comprising a ring-like geometry, and covering 120 which surrounds (wraps around) the perimeter of ring 110 and the circular area within the ring, as shown. Electronics module 170 is positioned on the distal end of implant 100. Sensor 150 is positioned proximate covering 120 as shown. Alternatively, sensor 150 can be positioned in a more distal portion of implant 100. In some embodiments, sensors 150 comprises one or more sensor oriented away from covering 120, such as to measure pressure within the LA while avoiding direct contact with blood circulating in the LA (e.g. reducing the likelihood of a reaction between a sensor of sensor 150 causing thrombus formation and/or a reaction between sensor 150 that deteriorates the performance of the sensor. Implant 100 can be of similar construction and arrangement as implant 100 described hereabove in reference to FIG. 1.

Implant 100 can include one or more anchors, anchor 140, which can comprise one or more microbraids and/or coils configured to anchor implant 100 and/or to cause stasis within the LAA.

Referring now to FIGS. 7A-C, a series of steps of implanting a device that electrically and fluidly isolates the left atrial appendage is illustrated, consistent with the present inventive concepts. In FIG. 7A, the LAA of a patient is shown. In FIG. 7B, implant 100 has been positioned such that the ring-like portion of ring 110 is positioned along the LAA ostium. Ring 110 can be configured with a resilient bias of sufficient radial strength to cause electrical isolation of the LAA. Alternatively, ablative energy can be applied to LAA ostial tissue via the ring-link portion of ring 110, as described herein, or the diameter of the ring can be increased, also as described herein. In FIG. 7C, fill material 130 has been injected into the LAA, also as described herein.

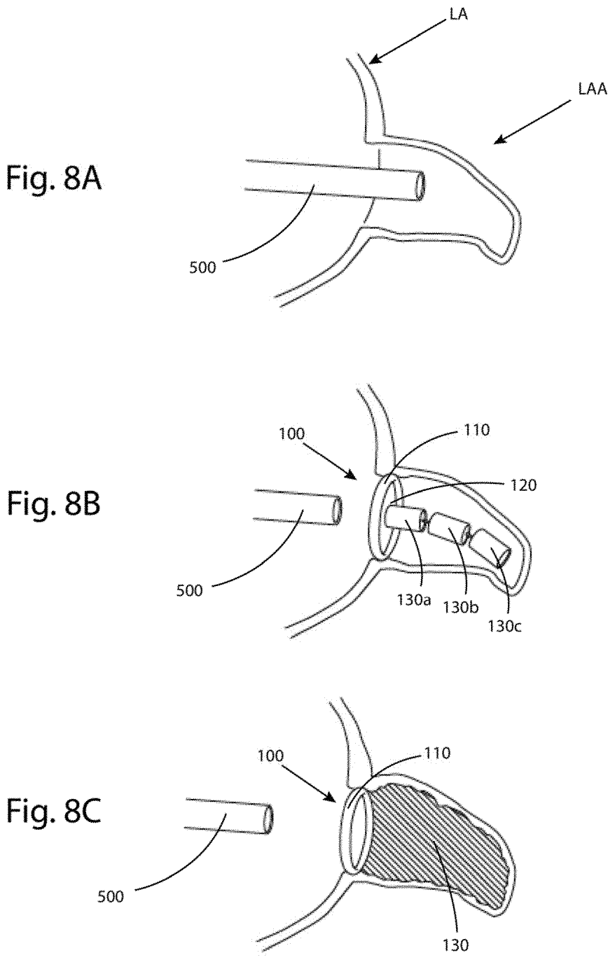

Referring now to FIGS. 8A-C, a series of steps that fluidly isolates the left atrial appendage is illustrated, consistent with the present inventive concepts. In FIG. 8A, the LA and LAA of a patient are shown, with delivery device 500 advanced into the LAA. In FIG. 8B, implant 100 has been deployed such that ring 110 engages the ostium of the LAA. Implant 100 includes multiple fill portions, such as fill material 130a-c shown. Fill material 130a-c can be configured to expand when contacting a fluid such as blood, as shown in FIG. 8C. Fill material 130a-c can be customized to properly fill the patient's LAA, such as to avoid underfilling or overfilling the patient's LAA. For example, imaging device 400 (as described hereabove in reference to FIG. 1) can produce one or more anatomical images of the patient's LAA from which fill material 130a-c can be customized. In some embodiments, fill material 130 comprises a particular number of portions based on the patient's LAA size and/or shape. Fill material 130 can comprise one portion, two portions, three portions (as shown), or four or more portions. Ring 110 can be deployed (e.g. expanded) prior to and/or after the expansion of fill material 130. In some embodiments, implant 100 is further configured to electrically isolate the LAA, such as when ring 110 applies a force upon tissue to cause the electrical isolation, and/or when electrically-isolating energy (e.g. RF energy) is delivered by ring 110 to the tissue, each as described herein.

FIG. 9 shows another embodiment of a system 10 configured to fluidly isolate the Left Atrial Appendage (“LAA”) from the left atrium (“LA”) to reduce risk of clot formation in the LAA and clot migration to the LA that might otherwise result (e.g. in a patient with AF or otherwise compromised LA function). Alternatively or additionally, system 10 can comprise an implant configured to electrically isolate the LAA (e.g. as a therapeutic treatment for AF). The system 10 includes an implant delivery device 500 having an elongate shaft, shaft 510, with a distal portion 511 configured to deliver an implant 100 to the LAA. The delivery device 500 may be one or more catheter-based delivery devices used in an interventional procedure (e.g. an over-the-wire interventional percutaneous procedure). In the alternative, implant 100 may be delivered to the LAA in a surgical procedure without a delivery device 500.

The implant 100 includes a frame or ring 110 configured to secure implant 100 to heart tissue near the ostium, a covering 120 surrounds at least a portion of ring 110 to fluidly seal the LAA, fill material 130 configured to be positioned within LAA, and one or more sensors 150 configured to send information from the implant. Optionally, the implant 100 may include an anchor 140 (not shown) configured to anchor the implant 100.

The ring 110 is configured to secure implant 100 to heart tissue. The ring 110 can include a ring-shaped configuration designed to engage the ostium (“OS”) of the LAA. The ring-shaped configuration may be an isolation ring configured to compress the OS tissue.

The covering 120 that surrounds at least a portion of ring 110. Covering 120 can include different embodiments and features, described herebelow.

The fill material 130, which can be positioned within LAA and may be one part or have multiple parts. FIG. 9 shows a multiple part fill material 130 configuration having three fill material sections, a proximal fill 130a, an intermediate fill 130b, and a distal fill 130c. The multiple fill material sections 130a-c may be the same size with same material composition or may be different sizes and material. Fill material 130 can include different embodiments and features, described herebelow.

An anchor 140 may comprise one or more anchoring elements (e.g. in addition to or as an alternative to ring 110 functioning as an anchoring element). Anchor 140 can include different embodiments and features, described herein.

One or more sensors, sensor 150, such as one, two, or more sensors selected from the group consisting of: an electrode; an electrical activity sensor; a heart rate sensor; a pressure sensor; a pH sensor; a blood sensor; a blood gas sensor; a chemical sensor; and combinations of one, two, or more of these. In some embodiments, sensor 150 is configured to transmit and/or record one or more patient physiologic parameters, such as blood pressure within and/or at least proximate the heart; blood flow velocity and/or direction; ECG; left atrial pressure; heart rate; and combinations of one, two or more of these. In some embodiments the sensor 150 wirelessly transmits information to an external system or device, such as a computer, tablet device, or smart phone. In some embodiments, the sensor 150 receives information to treat one or more patient medical conditions.

One or more portions of implant 100 can be configured to unfold and/or otherwise expand during and/or after deployment. In some embodiments, implant 100 is configured to be recaptured during and/or after implantation (e.g. during and/or after deployment from delivery device 500).

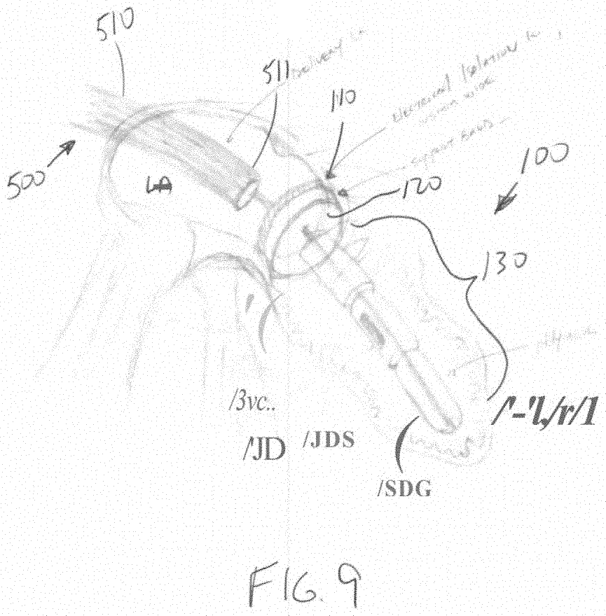

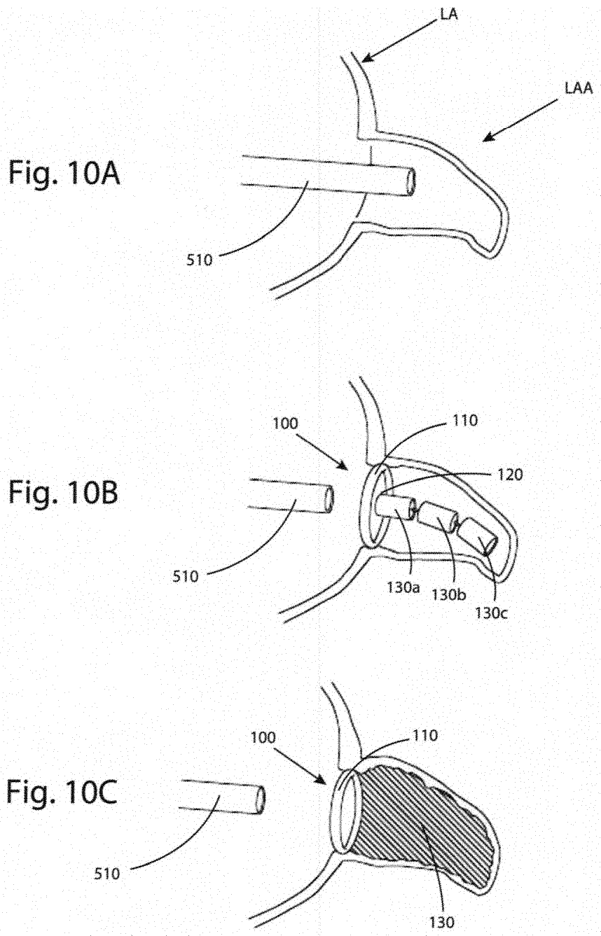

Referring now to FIGS. 10A-10C, a series of steps that fluidly isolates the left atrial appendage is illustrated, consistent with the present inventive concepts. FIG. 10A, shows the delivery device 500 advanced into the LA toward the LAA. FIG. 10B shows implant 100 implanted, with the fill material 130 being deployed within the LAA and the ring 110 engaging the OS. In the embodiment shown, fill material 130 includes multiple fill material sections 130a-c that are coupled together. Fill material 130 can be configured to expand within the LAA, as shown in FIG. 10C. Fill material 130 can be customized to properly fill the patient's LAA, such as to avoid underfilling or overfilling the patient's LAA. For example, an imaging device may be used produce one or more anatomical images of the patient's LAA from which fill material 130 can be customized. In some embodiments, fill material 130 comprises a particular number of fill material sections based on the patient's LAA size and/or shape. Fill material 130 can comprise one section, two sections, three sections (as shown), or four or more sections. Ring 110 can be deployed (e.g. expanded) prior to and/or after the expansion of fill material 130. In some embodiments, implant 100 is further configured to electrically isolate the LAA, such as when ring 110 applies a force upon OS tissue to cause the isolation.

As described above, the implant 100 is configured to engage the LAA and includes ring 110 configured to engage the ostium, covering 120 surrounding at least a portion of ring 110, and fill material 130 configured to fill the LAA. The implant 100 is configured to fluidly isolate and/or electrically isolate the LAA to reduce the risk of embolization, such as to reduce the risk of clot formation and migration out of the LAA,

The frame or ring 110 is configured to apply a force to the ostium (e.g. a force exerted fully circumferentially about the LAA ostium) to disrupt cell to cell conduction within the tissue (e.g. by compressing myocytes to prevent ion exchange) that results in electrical isolation of the LAA. The frame or ring 110 is configured to expand and stretch cardiac tissue to disassociate the myocytes. In some embodiments, the ring 110 provides at least a 2:1 stretch to the cardiac tissue to disassociate the myocytes. So, a 15 mm LAA ostium will need a 30 mm ring compressed and implanted,

Over time, a biological response occurs (e.g. as a result of the pressure and slight stretching caused by implant 100) that results in endothelial cell formation and fibrous tissue growth in the LAA ostial tissue. The cellular matrix created by implant 100 is non-conductive, providing long term electrical block. In some embodiments, ring 110, an isolation ring, and/or another portion of implant 100 is configured to deliver ablative energy to tissue, such as thermal energy (e.g. ablative heat or ablative cooling), radiofrequency (RF) and/or other electrical energy (e.g. ablative electrical energy), and/or sound energy (e.g. ablative ultrasound energy), such as during delivery when implant 100 is temporarily connected to a source of energy (e.g. electrical, thermal, and/or sound energy provided by energy delivery unit 600).

In some embodiments, the ring 110 includes a ring-like portion or isolation ring having an adjustable geometry, such as an adjustable diameter. In some embodiments, the ring 110 is positioned in the LAA ostium, and the diameter of the ring-like portion is increased until electrical isolation of the LAA is achieved (e.g. due to the effect on the tissue described hereabove).

FIG. 10D shows one embodiment of a split ring 110 having an adjustable geometry. The split ring includes a locking mechanism 114 that allows the ring to increase in diameter to engage the tissue. The locking mechanism 114 can comprise a ratchet geometry whose diameter can incrementally increase (e.g. via a ratchet-control mechanism).

In other embodiments, the ring-like portion of ring 110 can comprise a shape memory material that changes dimension (increases diameter) in response to an application of energy (e.g. energy delivered using delivery device 500).

FIGS. 11A and 11B show one embodiment of a LAA isolation ring 110 configured to apply maximum pressure to the LAA having a band, inner ring 110a with an outer raised isolation portion, outer ring 110b. in this case the pressure is applied near the ostium OS. The raised tab, outer ring 110b creates a small area where the pressure is applied. In the embodiment shown, the band, inner ring 110a has a first width W1 for stability while the outer ring 110b has a second width W2 for maximum pressure. In some embodiments, the inner ring 110a may have a width approximately 5 mm and the outer ring 110b may have a width between 1-5 mm. In some embodiments, the outer ring 110b may have a localized width <5 mm of LAA ostial tissue and the inner ring 110a may have at least 2× the width of the outer ring 110b for implant stability. The LAA isolation ring 110 is configured to deliver pressure 360 degrees. In some embodiments, the ostium may not be round and the outer ring 110b may have a variety of shapes, such as an oval shape. These non-circular shapes create a design challenge to develop an ostium ring which delivers adequate/equal pressure at all points around the ostium.

FIGS. 11C and 11D show another embodiment of a LAA isolation ring having an inner ring 110a and an outer ring 110b with two diameters configured to apply maximum pressure to the OS of the LAA. The outer ring 110b has a larger diameter and is configured to apply pressure to the OS tissue. The inner ring 110a has a smaller diameter but is also wider than the outer ring 110b to provide isolation ring 110 stability post-implant. The inner ring 110a may also be configured to allow for energy to be delivered to the LAA tissue via an ablation catheter. In some embodiments, the ostium may not be round, and the inner ring 110a and outer ring 110b may be made of a flexible material to allow the isolation ring 110 to conform to the ostium and deliver adequate/equal pressure at all points around the ostium.

In some embodiments, a device that does both LAA occlusion with electrical isolation (i.e. one device for both functions). The device is configured to apply pressure to a select area of tissue around the LAA ostium (Ostia isolation ring), such that the pressure prevents the myocytes sodium/potassium ion exchange to occur, this renders the myocytes in-active (non-conductive), resulting in electrical block.

The same device also creates a barrier between the left-atrium and the left atrial appendage, blocking any blood flow into or out of the LAA.

The same device could also be connected to a fill material that is deployed inside the LAA to absorb blood and expand into the LAA space, preventing blood flow into or out of the LAA.

The same device could also have a semi-conductor covering (bio-sensor) to measure and record bio-data, such as pressure, temperature, electrical activity, blood flow, etc.

Semi-conductor material could act as a cover 120 for the space inside the ostial isolation ring, the semi-conductor could also be implanted on the wall of the heart, slightly embedded into the heart wall as to not stand above the tissue level and create a potential clotting issue.

If embedded in the wall, the semi-conductor could also measure and record heart wall motion, etc.

The semi-conductor could be designed in a way to be implanted over the mitral valve, while measuring and recording bio-data as mentioned above. The semi-conductor could trigger a valve, or several valve-like features, incorporated into the design to open and close as needed each heartbeat.

FIG. 11E shows the isolation ring, inner ring 110a providing electrical isolation, disconnecting the LAA from the left-atrium to improve AF ablation procedures and/or to reduce AF burden from arrhythmogenic foci coming from the LAA. The raised isolation tab, outer ring 110b may be used for ablation while the inner ring 110a provides stability.

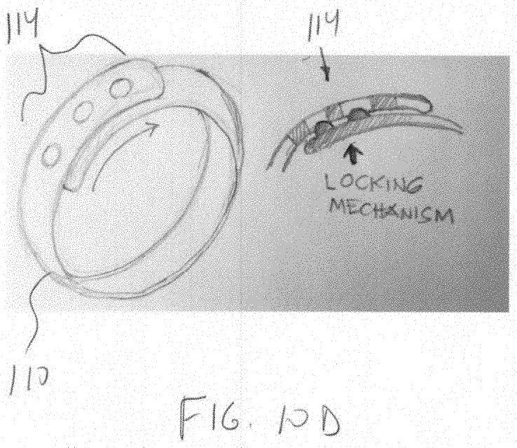

FIGS. 12A and 12B show an embodiment of an Isolation Ring 110 with Left-Atrial Appendage Cover 120. The isolation ring 110 has a first portion 110a having a diameter for maintaining positioning, and a second portion 110b having a larger diameter for an electrical conduction block. The outer edges of the first portion 110a are designed to engage adequate LAA tissue such that the outward force holds the cover 120 in place. The second portion 110b is designed to create electrical conduction block between the Left-Atrium and the Left-Atrial Appendage by the outward force which will compress the myocytes.

FIG. 13 shows another embodiment of an electrical isolation ring 110 that engages the cardiac tissue near OS with a biocompatible material-embedded carbon nanotubes (“BC-ECN”) to help create a line between the LA and the LAA. This design could recreate a conduction pattern that would allow for circular conduction 155 but not allow conduction across the line of the ring 110, thereby preventing signals from LAA going into LA.

The ring 110 may include a layer that applies pressure to the LAA ostium with BC-ECN. The carbon nanotubes would create a line around the ostium that would direct any signal coming into it into a circular pattern. The conduction pattern would allow for circular conduction but not allow conduction across the line between the left atrium and the LAA and prevent signals from LAA going into the LA.

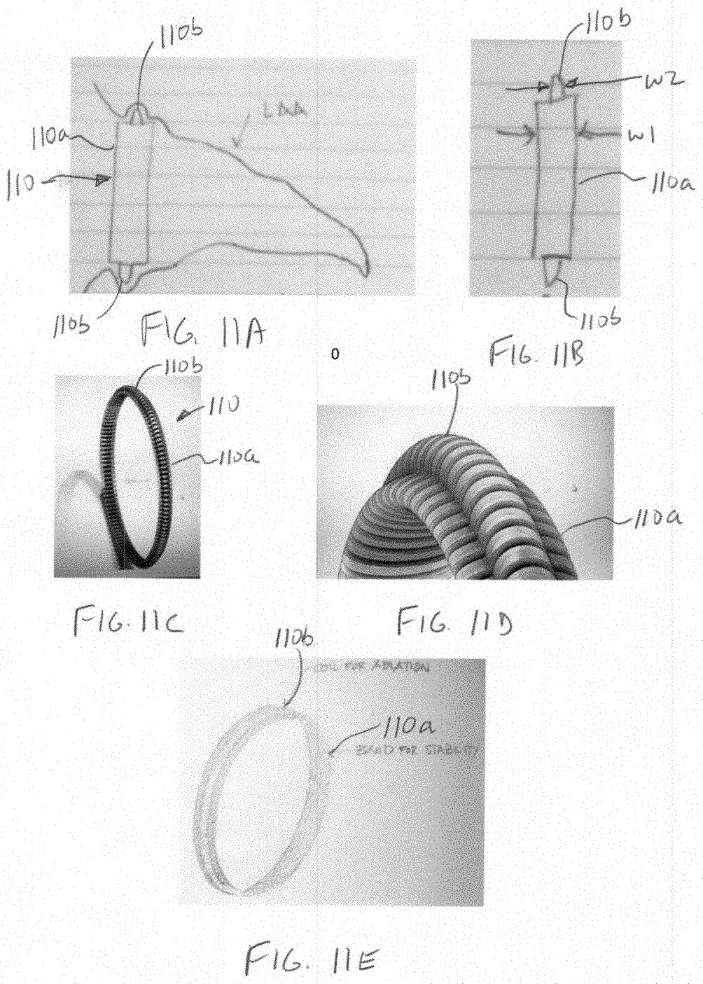

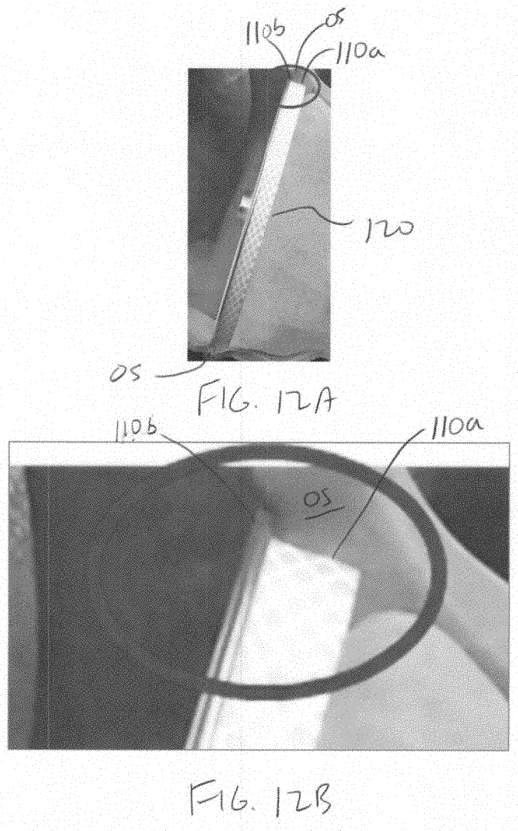

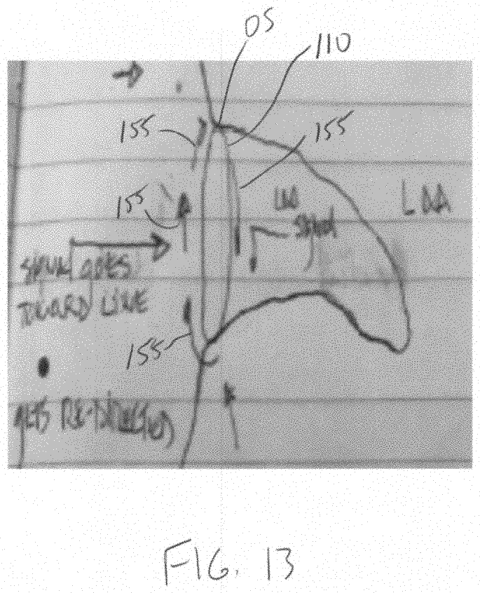

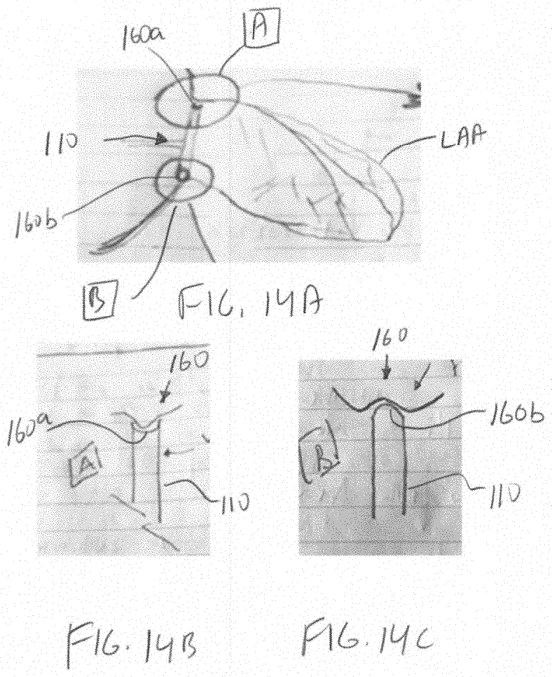

FIGS. 14A-14C show another embodiment of a LAA ring 110 that provides the combination of pressure mediated ablation plus electrical energy ablation.

The outer most portion of the ring 110 will have a circular/ring shaped component 160 that will have a feature to create a vacuum to pull the LAA ostial tissue to a convex state 160a, shown in FIG. 14B. Once LAA ostial tissue is pulled into the convex state 160a due to vacuum, an electrical current is delivered around the isolation ring 110 to the small width (e.g. ≤10 mm) of LAA ostial tissue in 360 degrees of LAA ostial tissue.

Once electrical current has been delivered, vacuum is discontinued, the device goes from a first convex state 160a to a second concave state 160b, shown in FIG. 14C. In the convex state 160a the ring 110 applies pressure to the tissue such that the pressure prevents the myocytes sodium/potassium ion exchange from occurring, this renders the myocytes in-active (e.g. non-conductive), resulting in electrical block.

LAA ostiums are not typically round, having a variety of shapes, such as an oval shape. These non-circular shapes create a design challenge to develop an ostium ring which delivers adequate/equal pressure at all points around the ostium.

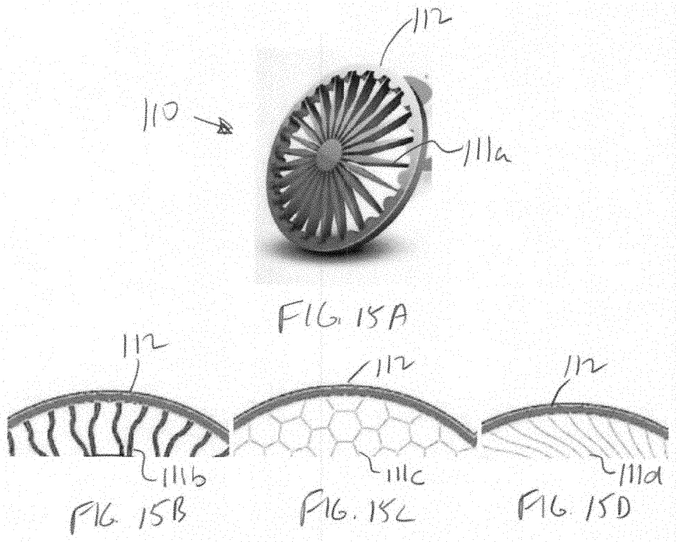

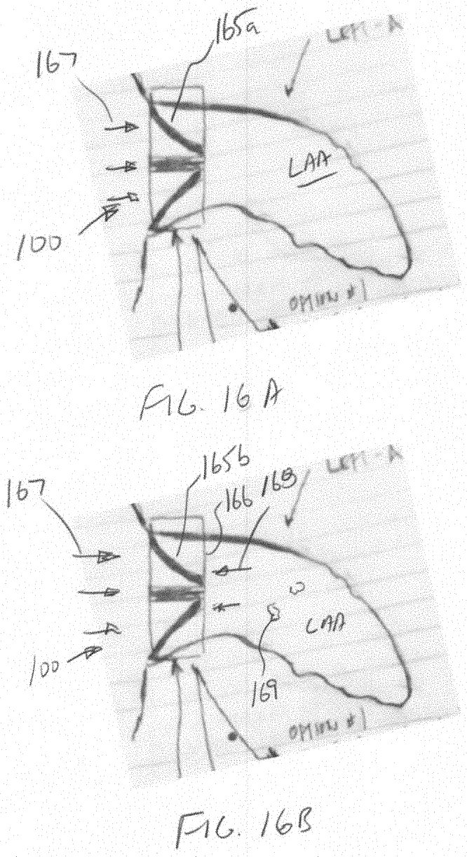

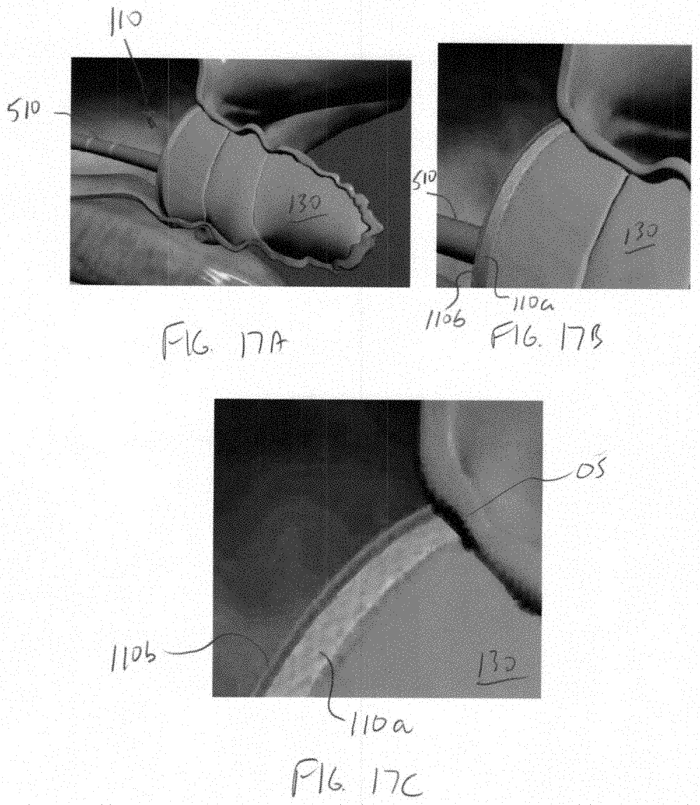

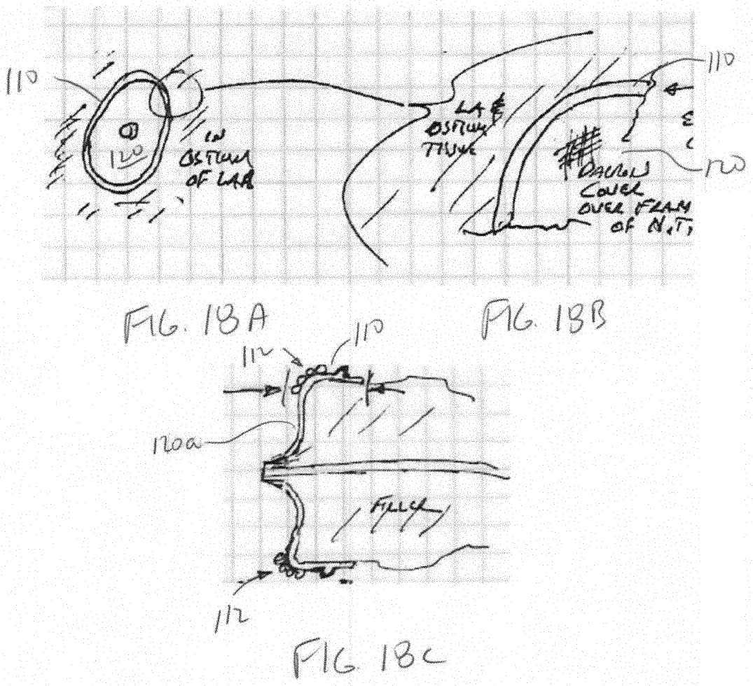

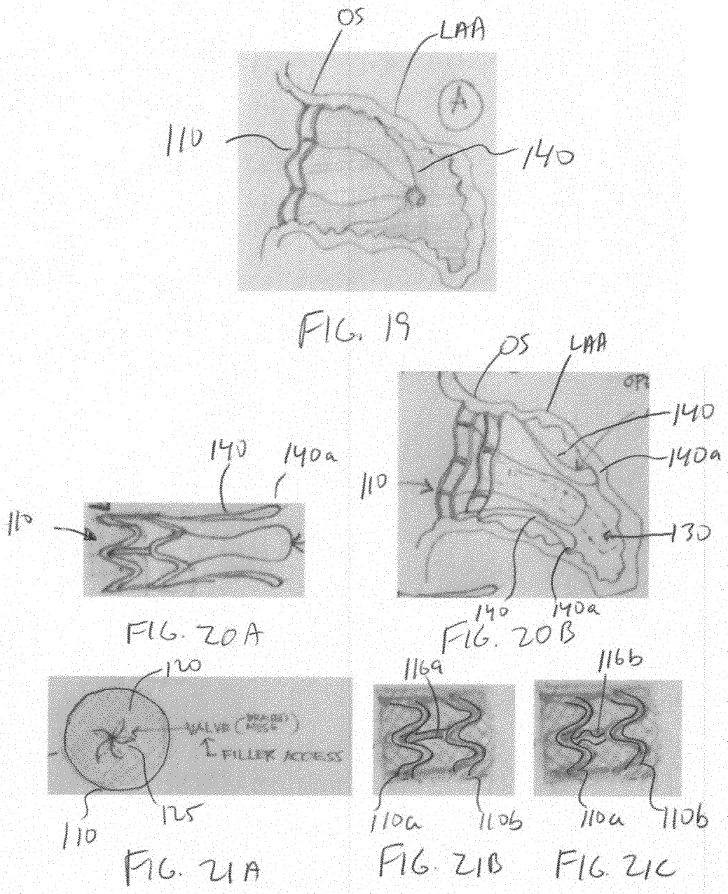

FIG. 15A shows one embodiment of Isolation Ring 110 or ostium ring having a plurality of spokes 111 surrounded by an outer ring 112. The spokes 111 are configured to flex and apply equal pressure around the outer ring 112 as the outer ring changes shape. The spokes 111 may be flexible radial spokes designed to bend while still providing constant tension to the outer ring 112, such as when the outer ring 112 changes shape to allow the isolation ring 110 to conform to the ostium and deliver adequate/equal pressure at all points around the ostium. The spokes 111 may be made of a flexible material that has both elasticity and stiffness at the same time. By varying the thickness and size of the spokes 111 they can have a wide range of flexible properties.

FIG. 15A shows the spokes 111a as radial spokes, but other spoke designs may be used to increase or decrease the flexibility of the design. FIG. 15B shows one embodiment of paired spokes 111b having two curvatures that alternate. FIG. 15C shows one embodiment of a honeycomb spoke 111c in which the number and size of the cells can vary for different mechanical properties. FIG. 15D shows one embodiment of a spoke 111d that is curved.

In some embodiments, the cover 120 may be configured as a barrier preventing blood from flowing into or out of the LAA. Cover 120 can be any suitable material to prevent blood flow, such as expanded PTFE, PTFE, woven polyester fabric, biocompatible materials, polyurethane membrane, etc. Cover 120 is secured directly to ring 110, such as by adhesive or stitching with suture material.

FIGS. 16A and 16B show embodiments of the implant 100 having one or more valves 165 positioned by the OS configured to control blood flow between the LA and LAA. In some embodiments, the valves 165 are one-way valves 165a allowing blood to flow in only one direction, and in other embodiments, the valves may be two-way valves 165b allowing blood to flow in both directions.

In some embodiments, the valve is a one-way valve 165a configured to allow blood flow into the LAA, but not allow blood flow out of the LAA, shown in FIG. 16A. In some embodiments, the valve 165a is configured to clot over time, forming a solid.

In some embodiments, it may be desirable to preserve LAA function to allow blood flow in and out of the LAA. FIG. 16B shows a valve that is a two-way valve 165b with a filter or screen 166 that is configured to allow blood flow in 167 and out 168 of the LAA while at the same time the screen 166 prevents clots 169 from coming out of LAA. This maintains LAA functionality while preventing clots 169 formed in the LAA from coming out and causing a stroke.

Implant 100 can comprise a cover 120 that surrounds at least a portion of ring 110. In some embodiments, cover 120 comprises a material selected from the group consisting of: a woven material; a fabric; a wire mesh; polyethylene terephthalate; a sponge; cellulose; synthetic fiber; cotton; rayon; hydrogel; a coagulant; a biodegradable material; a non-biodegradable material, and combinations of one, two, or more of these.

One or more portions of implant 100, such as cover 120 and/or fill material 130 can be configured to fluidly isolate the LAA, as described herein. In some embodiments, ring 110 engages the LAA ostium, and cover 120 provides a complete seal between the LA and the LAA, such as is described herebelow.

FIGS. 17A-17C show one embodiment of an implant 100 having a cover 120 that includes a self-expanding, disc-like structure, to close the LAA at the ostium's circumference and provide a smooth surface for endothelialization of the cover 120 over the LAA ostium. The cover 120 is placed to avoid any areas of turbulent blood flow to minimize the potential for thrombus formation on the LA-side surface. The cover 120 may be constructed with a Dacron or ePTFE membrane.

In some embodiments, the cover 120 is “flexibly attached” to the fill material 130 for retention while providing the implanting physician an ability to place the cover at an obtuse angle to the axis of the fill material 130 (as many of the LAA ostia are not orthogonal to the axis of the LAA shape). The edge of the cover uses two distinct points of contact with the LAA ostium to enable two electrical pathway isolation features, acute and chronic.

Acute isolation may be achieved through radio-frequency ablation energy passing through an electrically conductive ring 110a at the most proximal on the cover 120 edge/fabric surface. The ring 110a is likely a soft conductive metal, such as gold or platinum, exposed and mounted to the surface of the cover 120 so that it is accessible by the tip of an ablation catheter to energize after placement.

Chronic isolation may be achieved through mechanical compression on the LAA ostium's tissue (myocytes) using a mechanical compression ring 110b adjacent to, but proximal on the edge of the cover 120, which can be separate from the lesion created by the gold ring. The mechanical compression ring 110b may be achieved by the expanding structure underneath the Dacron or ePTFE cover surface. The cover 120 will somewhat reshape the LAA ostium to impart radial pressure which in 30-45 days post implant will achieve electrical isolation by compressing the tissue myocytes.

In some embodiments, the electrically conductive ring 110a and the mechanical compression ring 110b may be the same ring that does both functions.

FIG. 18A shows one embodiment of a multi-functional ring 110/cover 120 positioned within the ostium of the LAA. FIG. 18B is an enlarged sectional view of the multi-functional ring 110/cover 120. The multi-functional ring 110/cover 120 includes the ability to develop the two functions, electrical isolation and mechanical isolation, independently.

The multi-functional ring 110/cover 120 includes a cover 120a placed over a wire/strut structure 110. The cover 120 may be constructed of Dacron (polyester) or ePTFE placed over a NiTi wire/strut structure 110—not unlike many expanding stents, etc.

FIG. 18C is a cross-sectional view of the multi-functional ring 110/cover 120 showing additional details. In the embodiment shown, multiple winds of an electrically conductive wire 112, e.g. gold wire, are captured over/on the Dacron cover 120, i.e, outside of the Dacron cover, to be in direct contact with the LAA tissue. The multiple winds 112 form a “ring” on the shoulder of the cover 120 that can expand with the cover as it deploys. These winds assure that the ring is continuously conductive around the circumference and in section provide a “proud” contact into the LAA ostium to prioritize the wire contact to the ostium tissue over the Dacron (the wire is taller than the Dacron surface in profile). The conductive wire 112 allows both direct contact with the tissue and exposure of the conductive wire on the shoulder face of the cover to facilitate contact with an ablation catheter. The multiple winds 112 of gold wire enables the feature for acute isolation through circumferential ablation (one or more contacts with the ablation catheter).

The wire/strut structure 110 is a NiTi structure that provides self-expanding mechanical forces within the cover to expand, and also provides the ability for the multi-functional ring 110/cover 120 to provide radial force on the ostium and compliance to fit the non-round shape of the ostium. The NiTi structure provides the features/functions for the radial expanding “compressive force” on the ostium tissue. The NiTi structure 110 is positioned adjacent but separate from the electrically conductive wire wrap 112 so that the radial “compressive force” is applied to native, non-lesion, tissue of the ostium. While formed from the NiTi structure, it generates a “proud feature” through the NiTi cover to exert this localized compression on the tissue.

Some embodiments of the invention include an anchor 140 comprised of one or more anchoring elements (e.g. in addition to or as an alternative to ring 110 functioning as an anchoring element). Anchor 140 can comprise one or more coils, such as is described in FIG. 3. Alternatively or additionally, anchor 140 can comprise one or more wire meshes, materials which absorb fluids, such as is described in FIGS. 4A-B.

Anchor 140 may comprise one or more microcoils, or one or more coils of similar construction and arrangement to treat an aneurysm, such as a brain aneurysm. Anchor 140 can engage tissue of the LAA to anchor implant 100.

Anchor 140 may comprise a wire mesh construction in a cylinder configuration and/or spherical configuration. Anchor 140 can be attached to ring 110 such that anchor 140 is positioned in the LAA. In some embodiments, an embolic agent is positioned within and/or around anchor 140 (e.g. fill material injected during the delivery of anchor 140). Anchor 140 may comprise single-layer or multilayer construction. Anchor 140 can be configured to provide rapid intraprocedural stasis (e.g. stasis of blood within the LAA).

Anchors 140 may be attached at one or more locations of ring 110. Anchors 140 may comprise materials selected from the group consisting of: nickel titanium alloy; stainless steel; a shaped memory material; a polymer; and combinations of one, two, or more of these.

FIG. 19 shows one embodiment of a ring 110 configured as a stent ring coupled with a basket type anchor 140 engaging the LAA. The stent ring 110 is delivered in a compressed or folded configuration and expanded proximate the LAA ostium, with the basket 140 extending into the LAA that may also anchor the device. A fill material may be injected into the LAA through the stent ring 110 to fill the LAA and surround the basket type anchor 140 to anchor the implant 100.

FIGS. 20A and 20B show another embodiment of a ring 110 configured as a stent ring coupled with one or more spline anchors 140 engaging the LAA. The stent ring 110 is delivered in a compressed or folded configuration and expanded proximate the LAA ostium, with the one or more spline anchors 140 extending into the LAA. The one or more spline anchors 140 include a first end coupled to the stent ring 110 and a second end having an engagement portion 140a configured to engage the inner wall of the LAA to anchor the implant 100. The engagement portion may include an outwardly curved portion configured to expand and engage the LAA when the expanding fills are positioned within the LAA and expanded. A fill material 130, such as expandable fills, may be inserted into the LAA through the stent ring 110 to fill the LAA and surround the one or more spline anchors 140.

FIG. 21A is a front view and FIGS. 21B and 21C are side views of another embodiment of a ring 110 configured as a stent ring comprising one or more sinusoidal rings 110a and a cover 120 configured as a braided mesh filter cover 120 with fill access 125. In the embodiment shown, the sinusoidal rings 110a comprise two sinusoidal rings and one or more connectors 116 between them. In FIG. 21B the connector 116a is shown as a straight connector and FIG. 21C shows a connector 116b that is sinusoidal.

The one or more sinusoidal rings 110a may be made of a self-expanding material, such as Nitinol, and configured to self-expand against the LAA tissue. The one or more sinusoidal rings 110a may be configured for ablation of the LAA tissue. In some embodiments, the sinusoidal rings 110a may be plated with a conductive material, such as gold or platinum plated. In some embodiments, a conductive coil or spring of gold or platinum may be threaded or wound around the sinusoidal rings 110a for ablation. In some embodiments, one or more conductive bands of gold or platinum may be placed or crimped on the sinusoidal rings 110a for ablation.

In some embodiments, the implant 100 further comprises at least one sensor 150. The at least one sensor can comprise one, two, or more sensors selected from the group consisting of: an electrode; an electrical activity sensor; a heart rate sensor; a pressure sensor; a pH sensor; a blood sensor; a blood gas sensor; a chemical sensor; and combinations of one, two, or more of these. The at least one sensor can be configured to record: ECG; left atrial pressure; heart rate; and combinations of one, two or more of these. The at least one sensor can be configured to measure the pressure at a location selected from the group consisting of: within the LA; within a pulmonary vein; and combinations thereof. In some embodiments, data collected by sensor 150 can be processed internally and/or by a device outside of the patient's body.

In some embodiments the one or more sensors 150 may be positioned on the inner and/or outer surface of cover 120. If positioned on the inner surface toward the LAA, the sensor measures LAA properties. If positioned on the outside surface toward the LA, the sensor measures LA properties. If a sensor 150 is placed on both sides, the LA and LAA properties will be measured. Implant 100 may include an electronics module of similar construction and arrangement to electronics module 170 described above.

In some embodiments, the one or more sensors 150 may be a pressure sensor that can also measure pressure waveform. For example, the one or more sensors 150 may be configured to measure LA pressure and also measure pressure waveform. The pressure waveform can be used to determine the rhythm: AF vs sinus. Therefore, with one sensor (pressure) both pressure and rhythm can be monitored.

In some embodiments, temperature measurement may be integrated into the pressure/waveform sensors and used as an indirect measure of cardiac output, or a flow sensor can be used to determine cardiac output. They may be more helpful than ejection fraction because it tells what the output is exactly.

FIG. 22 shows one embodiment of a system having an implant 100 with one or more sensors 150 configured to continuously monitor physiological data (e.g. ECG signals) and/or wirelessly transmit physiological data to an external system or device, such as a computer, tablet device, smart phone, etc. The sensor 150 may include a battery, such as a rechargeable battery that can be charged using known charging means, such as electromagnetic induction.

In some embodiments, the one or more sensors 150 may include multiple ECG leads configured to wirelessly transmit ECG data of the heart wirelessly to an external system or device. The data may be wirelessly transmitted in real-time, or it may be stored and wirelessly transmitted only when requested, for example while at the doctor office.

The sensors 150 may include many of the features and designs of the LP1100 LS Patch made by LifeSignals and LC100 Life Signal Processor (LSP) Platform (www.lifesignals.com). Physiological data can be captured and processed within the sensor and wirelessly transmitted to a communication device 200 having one or more destination receivers 275 to receive the physiological data for display and/or analysis within communication device 200, electronics module 270, such as shown in FIG. 22. Physiological data may include multi-lead ECG, respiration rate and ECG derived heart rate. In some embodiments the sensor 150 communicates via a receiver device 175 which forms a “wireless cable” between the sensor 150 and destination receivers 275. For ambulatory, remote monitoring and cloud-based applications, the sensor 150 can communicate with Wi-Fi enabled devices (such as smart phones, tablets and access points) directly via built-in Wi-Fi connectivity. Multiple radio types for robust wireless connectivity in congested network environments may be used that combine narrowband (Wi-Fi/MBAN) and ultra-wideband (UWB) radios that dynamically switch modes to reliably maintain a consistent wireless link between the sensor 150 and the receivers.

In some embodiments, the implant 100 further comprises an electronics module 170. The electronics module can be configured to: collect and/or process information; store information; transmit information; receive energy; and/or transmit energy. The implant 100 can further comprise at least one sensor 150, and the electronics module 170 can comprise an algorithm configured to analyze data recorded by the at least one sensor.

In some embodiments, the fill material 130 can be configured to absorb blood and/or other fluids present in the LAA. In some embodiments the fill material 130 is configured to expand to approximate the space of the LAA. The fill material 130 can be biodegradable, non-biodegradable, or a combination of biodegradable and non-biodegradable. The fill material 130 can comprise multiple fill material sections, for example 130a, 130b and 130c shown in FIGS. 1 and 2B. The fill material 130 can include at least one of radiopaque material or a radiopaque marker.

Fill material 130 can comprise a material selected from the group of biocompatibles consisting of: a sponge material; a fabric material; cotton; rayon; a hydrogel; a polymer; and combinations of one, two, or more of these.

Alternatively or additionally, fill material 130 can comprise an injectable material, such as an injectable embolization material such as polyvinyl alcohol (PVA). In some embodiments, fill material 130 comprises a material that biodegrades over time, such as a time of at least 1 week, at least 1 month, or at least 6 months, and/or a time of no more than 1 month, no more than 3 months, or no more than 6 months.

In some embodiments, the fill material 130 is an injectable embolization material or other material configured to fill the LAA. Fill material 130 can comprise an adhesive material. Fill material 130 can be used if cover 120 does not provide a complete seal between the LA and the LAA.

In some embodiments, the fill material 130 can comprise a geometry customized based on the patient's anatomy. The fill material can be customized to properly fill the patient's LAA, such as to avoid underfilling or overfilling the patient's LAA. For example, an imaging device can produce one or more anatomical images of the patient's LAA from which fill material 130 can be customized. In some embodiments the fill material includes a radiopaque material or marker and is monitored with an imaging device during and/or after delivery to the LAA. In some embodiments, fill material 130 comprises a particular number of fill material sections based on the patient's LAA size and/or shape. Fill material 130 can comprise one section, two sections, three sections (as shown in FIG. 9), or four or more sections. Ring 110 can be deployed (e.g. expanded) prior to and/or after the expansion of fill material 130.

In some embodiments, the fill material 130 can comprise LAA Polymer fill-material. Filling the LAA centers on an elastic, porous fabric (electro-spun fibers, hydroentangled to a nonwoven felt of high surface area), compressed and adhered together with an anhydrous hydrogel. The hydrogel may have tissue-adhesive properties.

After inserting the composite into the LAA, capillary action causes blood absorption. Water from the blood diffuses into the hydrogel, swells it and reduces its mechanical strength thereby allowing the fibrous network to expand. The blood constituents, presumed to be negatively charged, should adsorb to the hydrogel surface.

The blood absorption by capillarity and the swelling of the hydrogel by water causes the composite to expand to its original, uncompressed, volume and, since now highly compliant, filling the appendage and lodging itself.

Cationic charges on the hydrogel facilitate adhesion of the hydrogel to the heart tissue (negatively charged). Shape memory could be designed into the composite to help conform to the shape of the appendage to arrest its movement after completed deployment in its swollen form. Pressure sensitivity for the hydrogel can be achieved by molecular modifications.

In some embodiments, the fill material 130 is a hydrogel fiber structure. Biocompatible hydrogels have been employed for years in medical devices as void fillers, in wound healing and contact lenses. Their ability to absorb water to swell and fill is an attractive attribute for multiple uses in medical devices. However, these materials are notorious for poor mechanical integrity and strength (before and after swelling in the presence of water), easily succumbing to tear and rupture in solo use.

This embodiment of fill material 130 leverages the swelling/void filling advantages of hydrogels while combining them with a reinforcing structure of textile fibers to achieve a suitable material to meet the requirements.

A fiber structure that is non-uniform, allowing fiber amount and orientation to be configured along the length of the device to facilitate functionality and performance in both the predeployed configuration (deliverability down a catheter) and deployed configuration after expansion.

Hydrogel application to the fibers in compression, to assist in maintaining the compressed shape for catheter deployment (e.g. an adhesive or stabilizer to help hold the compressed shape of the fibers).

Hydrogel application that is non-uniform allowing for different hydrogel amounts or different type of hydrogels used along the length of the compressed fiber structure. Hydrogels are impregnated into the fiber structure along its length, e.g. the “filler” is formed from a combination of a fiber structure that is impregnated with anhydrous hydrogel. The fiber structure could be loosely woven (included loosely braided, or fabric like) or non-woven with fibers in a “lay-up” configuration. Fibers could be of several different materials but may include either poly(ethylene terephthalate), e.g. Dacron, or segmented poly(ether/estedurea/urethane), e.g. Lycra or Spandex that provide an elastomeric character, or both. The fiber structure will exhibit a sponge-like character due to the space, or porosity between the fibers. Fibers would be formed using an electrospun or equivalent process to result in very small fiber diameter, measured in microns. Given its low density, the fiber structure is also highly compressible to enable a pre-deployed configuration suitable for passage through a catheter lumen, and then expand into the LAA upon deployment wherein it may be configured to represent 10 to 20%, or more, by volume of the Guardian filler after deployment, the remaining volume being the hydrogel and likely some small amount of void space. The void space may be greater at one end of the Guardian device than the other—allowed by the design's performance, e.g. the low void space of possibly <10%, may be closer to the distal end to support the anchoring function or may be closer to the proximal end (or ostium) to assure effective sealing against the LAA wall with the other end allowing a larger amount of void.

The filler is prepared with the fiber structure, e.g. sponge, compressed in the radial and possibly the longitudinal direction. The fibers are arranged to achieve the fiber density and distribution as deemed appropriate for the device's performance before and after deployment. The compressed fiber structure is then impregnated with an aqueous solution, or solutions, of a hydrogel with the hydrogel(s) being minimally crosslinked to support flexibility and expansion, possibly containing 1-2% crosslinking agent. This composite is dried to remove the moisture wherein the dried hydrogel acts like an adhesive and prevents the compressed fiber structure from recovery (expanding) prior to deployment. The hydrogel impregnation and drying step may have to be repeated multiple times to achieve a sufficient fill of dried hydrogel in the interstitial spaces between the fibers. The presence of a small amount of chemical additives, such as benzophenone, in the composite could facilitate interfacial bonding between the fibers and the hydrogel. The dried fiber/hydrogel structure is then exposed to ionizing radiation, e.g. gamma or electron beam radiation, to effect crosslinking. The amount of radiation may be set to concurrently achieve sterilization of the device if properly configured with the rest of the device and deployment system and packaged for terminal sterilization.

Upon exposure to blood, the blood is drawn into the interstitial spaces of the fiber and the moisture from the blood is absorbed into the hydrogel. The fibers expand, like a sponge, to expose more surface area such that the hydrogel can absorb more moisture and accelerate further expansion and swelling of the composite. This combined swelling mechanism allows for a tailored approach to expansion wherein different combinations of fiber and hydrogel by volume %, can generate different rates of expansion to facilitate the device's deployment and ease of clinical use. The use of elastomeric fibers, e.g. Lycra, may facilitate greater mechanical integrity of the composite during expansion versus the potential for the hydrogel/fiber bond to fracture due to the highly expansive nature of the hydrogel, e.g. closer approximation of expansion coefficients, however the contractile force of the Lycra could inhibit the expansion of the hydrogel wherein a non-elastomeric fiber, e.g. Dacron, may not inhibit the hydrogels expansion as much. The selection of fibers would be determined by evaluation and testing to determine which composite achieves the product requirements.

Based upon the hydrogel chemistry employed, the hydrogel can be tuned to be pressure responsive. In this configuration, the fibers absorb the blood and can only swell to an initial amount until saturated. Concurrently, the hydrogel is swelling by absorption of the moisture in the blood. The hydrogel can be tuned such that when it experiences a specific compressive pressure, e.g. when it swells to fill the available volume and then generates an internal pressure, the mechanism for moisture uptake and further swelling is diminished resulting in a self-regulating expansion and minimum risk of injury or rupture to the LAA.

With this system, the filler is a solid device in the package, e.g. no preparation of intraoperative liquids and no requirement for in-situ polymerization. Once inserted into the LAA, the filler swells in blood; the rate of initial swelling is defined by the presence of fiber and the porosity in the composite device. Concurrently, the hydrogel absorbs moisture and swells. When the swollen hydrogel/fiber composite touches the LAA wall and generates an acceptable expansive force, absorption/swelling stops. The acceptable expansion and expansive force may be different along the length of the device, e.g. along the length of the LAA, to facilitate different functions of the filler, e.g. anchoring versus sealing.

Suitable hydrogel polymers are commercially available in high purity and high molecular weight, the requirements for biocompatibility. Further there are suitable multifunctional, crosslinking agents commercially available—when used in combination with the polymer and crosslinked in the presence of ionizing radiation, resulting in a biocompatible structure even though these crosslinkers may not be biocompatible in their as used condition. It's also possible that the hydrogel polymer may cross link under ionizing radiation sufficiently that a crosslinker may not be required in the composite to achieve the performance noted above. Note the selection of hydrogels and potential crosslinkers is based upon a preference for incorporating carbon-carbon bonds which prevents degradation of the composite structure by hydrolysis during assembly or during deployment and expansion within the LAA. The composite structure must maintain mechanical integrity through the implant's life expectancy, e.g. the patient's life expectancy.

Suitable hydrogel polymers may include PVA (poly(vinyl alcohol)) and its acetal derivatives, PNVP (poly(N-vinyl-pyrrolidone)), PEG (poly(ethylene glycol)), etc. The final selection would be determined by evaluation and testing to demonstrate the composite structure meets the device requirements.

The fill material 130, which can be positioned within LAA and may have one part or have multiple parts. Referring again to the embodiment shown in FIG. 9, the fill material 130 includes three fill materials: a proximal fill 130a, an intermediate fill 130b, and a distal fill 130c. The multiple fill materials 130a-c may be the same size with same material composition, or the implants may be different sizes and material. Fill material 130 can include different embodiments and features discussed herein.

Delivery device 500 can comprise one or more catheters or other tools configured to deliver a fill material 130 to the LAA. The fill material 130 can be configured to absorb blood present in the LAA and expand to approximate the space of the LAA. Since the fill material 130 is expandable when contacted with a liquid, the fill material should be delivered in a dry state so that it does not expand prior to placement in the LAA.

FIG. 23 shows one embodiment of delivery device 500 configured to deliver the fill material 130 to the LAA in a dry condition. The delivery device 500 includes a thin-walled tube applicator 520 having a closed distal end 521 coupled to a distal end 511 of a catheter shaft 510. Once in position within the LAA, the applicator 520 is configured to open to allow for delivery of the dry fill material 130. In the embodiment shown, the applicator 520 is a cylindrical shaped tube with a closed distal end 521 and a proximal end 522 coupled to the distal end of catheter shaft 511. The distal end may be a balloon-type distal end.

For deployment of the fill material 130, the closed distal end 521 of the applicator 520 is configured to open during delivery of the fill material 130. In the embodiment shown, the distal end 521 includes a plurality of skives 525 creating weakened areas designed to tear open when the fill material 130 is pushed out of the distal end 521. In other embodiments, distal end 521 may be manufactured with weakened sections or areas for tearing. In some embodiments, the distal end 521 may include flaps or other types of openings to allow delivery.

For delivery, the distal end of delivery device 500 with applicator 520 is advanced to the LAA. The fill material 130 is inserted through the catheter and makes contact with the closed distal end 521 of the tube applicator 520. The skives 525 on the distal end tear open and allow the fill material 130 to be pushed out of the distal end 521. The dry fill material 130 makes contact with the blood and absorbs the blood to expand within the LAA.

In some embodiments, the tube applicator 520 may be configured to close once the fill material 130 is dispensed from the distal end 521. While closed, a second fill material section of dry fill material 130 may be advanced within the catheter shaft 510 and contact the distal end 521 and open the skives 525 to be pushed out of the distal end 521. This filling process can continue for a 3^{rd}, 4^{th} or any number of fill material sections of dry fill material 130 to be delivered.

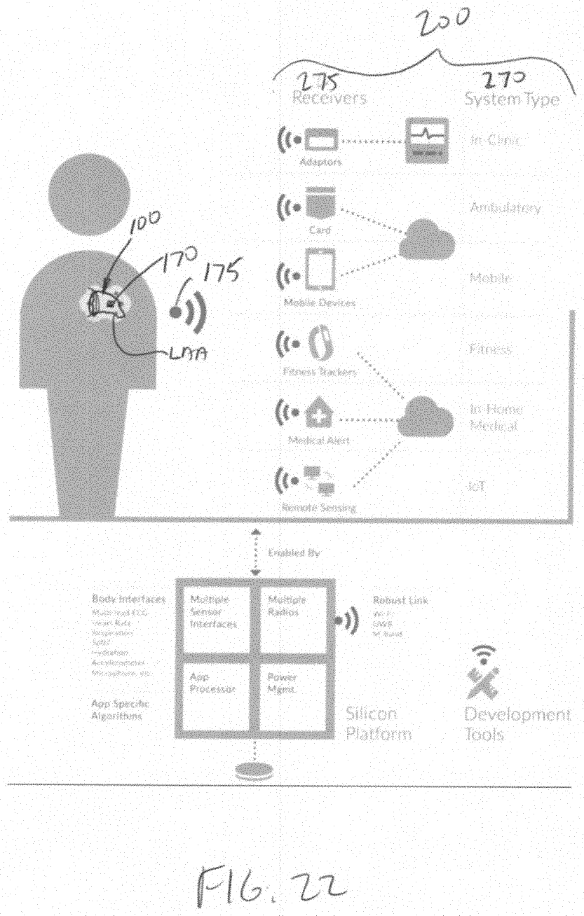

FIG. 24 shows another embodiment of fill material 130 configured to absorb blood when located in the LAA and expand to fill the LAA. The fill material 130 is configured to be twisted like a rope during delivery to prevent fluid absorption prior to placement in the LAA. The twisted shape is sized to slide through a catheter for delivery into the LAA. Once the twisted fill material 130 is positioned in the LAA it will absorb blood and expand to approximate the space of the LAA to prevent the blood forming clots which could then cause stroke in patients where the clots come out of the LAA and are pushed to the brain by the heart's pumping of blood.

In some embodiments, the twisted fill material may be delivered through the applicator 520 as described hereaabove in reference to FIG. 23.

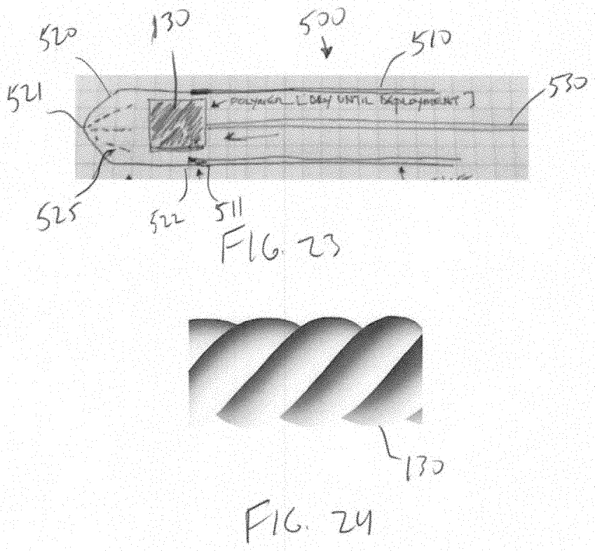

FIGS. 25A-25C show one embodiment of a fill material 130 being delivered into the LAA. The fill material 130 may be one section, or multiple sections. In the embodiment shown, the fill material 130 comprises three fill material sections 130a, 130b, and 130c. Once delivered, the fill material sections 130a-c are configured to absorb blood in the LAA and expand to fill the LAA. The filler or fill material 130 may be a polymer/foam/other (i.e. material) that is delivered to the LAA and expands via absorbing blood into the various trabeculations, peaks and valleys of the LAA anatomy.

In some of the embodiments, the fill material sections 130a-c may be the same configuration, having the same length and same expanded diameter. In other embodiments, the fill material sections 130a-c may be different configurations, having different lengths and/or different expanded diameters. In some embodiments, the fill material sections 130a-c are made of the same material with the same material properties. In other embodiments, the fill material sections 130a-c are made of different materials and may have different properties. The properties may include both mechanical properties and electrical properties.

FIG. 25A shows the distal fill material 130c being delivered from the distal end 511 of a delivery device 500. Fill material 130c is shown being delivered using a wire component 530.

FIG. 25B shows distal fill material 130c in the expanded state after absorbing blood in the distal portion of the LAA. The figure also shows intermediate fill material 130b being delivered from the delivery device 500 and contacting the blood and starting to expand.

FIG. 25C shows the distal fill material 130c and intermediate fill material 130b in the expanded configurations after absorbing blood within the LAA. Proximal fill material 130a is shown exiting the distal end 511 of the delivery device 500 and contacting the blood and starting to expand.

This expansion of the fill material 130 fills the space previously occupied by the blood within the LAA. The expansion of the fill material 130 may generate a radial force on the inside of the LAA. The combination of a soft material surface capturing the trabeculations of the inner LAA structure and broad area radial force, as demonstrated by slight expansion of the LAA, will provide a suitable anchoring function to retain the implant. This compliant structure and slight expansion also minimizes post implant thrombus formation by completely filling the native LAA, completely sealing the ostium/entrance to the native LAA from LA blood access.

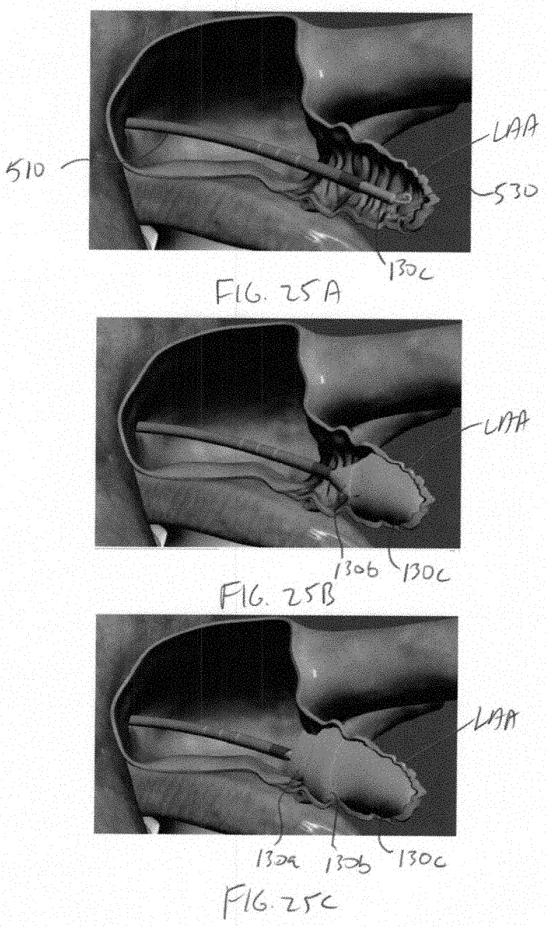

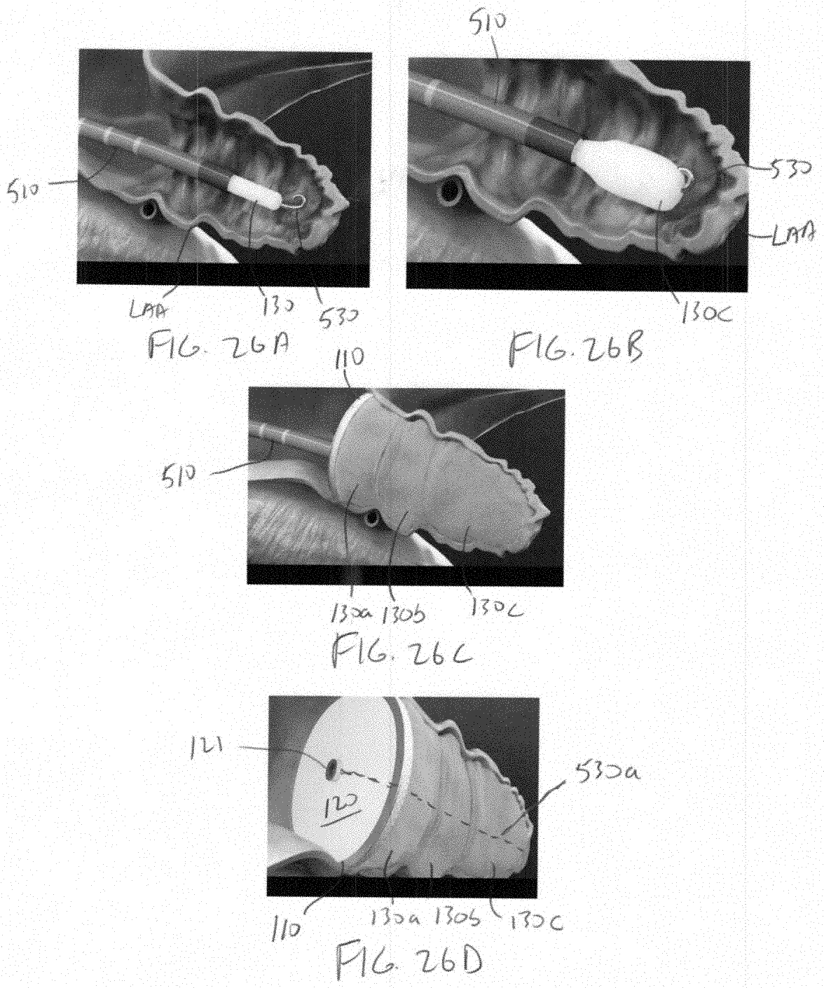

FIGS. 26A-26D show one embodiment of an implant 100 delivered to the LAA. The implant includes a cover 120 and fill material 130, delivered in sections 130a, 130b, and 130c, and a center lumen/wire 530 connecting the fill material sections 130a-c to the cover 120. In some embodiments, the cover 120 may be an isolation ring 110/cover 120, as described herein. Once delivered into the LAA, the fill material sections 130a-c are configured to absorb blood in the LAA and expand to fill the LAA

In some of the embodiments, the fill material sections 130a-c may be the same configuration, having the same length and same expanded diameter. In other embodiments, the fill material sections 130a-c may be different configurations, having differed lengths and/or different expanded diameters. In some embodiments, the fill material sections 130a-c are made of the same material with the same material properties. In other embodiments, the fill material sections 130a-c are made of different materials and may have different properties. The properties may include both mechanical properties and electrical properties.

FIG. 26A shows initial delivery of a first or distal section of fill material section 130c into the LAA through a delivery device 500 using center lumen/wire 530. As the fill material 130c is delivered from the distal end of the delivery device 500, it starts to absorb blood and expand, as shown in FIG. 26B. Each subsequent fill material section 130 will also exit the delivery device 500 and absorb blood and expand.

The fill material sections 130a-c may be positioned close together and delivered at the same time, or the fill material sections may be spaced apart to deliver and expand one at a time.

FIG. 26C. shows the implant 100 with the multiple fill material sections 130a-c expanded in the LAA. After the fill material 130 is delivered, a cover 120 is then delivered and positioned within the LAA ostium.

FIG. 26D shows a proximal view of the implant 100 after the delivery device 500 is disconnected from the cover 120 at a catheter/cover connection 121.

In some embodiments, the center lumen/wire 530a terminates on the distal side of the cover 120 and remains within the implant 100, connecting the cover 120 with the multiple fill material sections 130a-c.

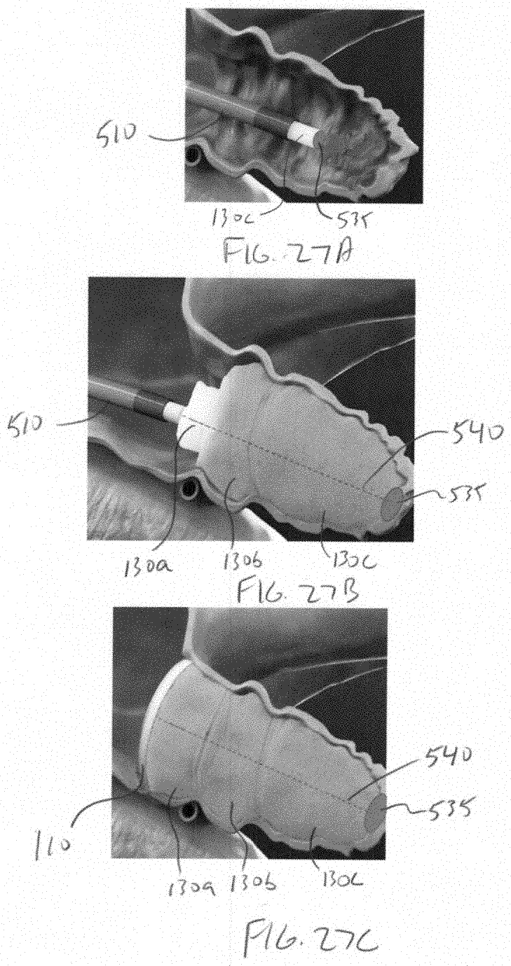

FIGS. 27A-27C show another embodiment of an implant 100 delivered to the LAA.

The implant includes a cover 120 and fill material 130, delivered in sections 130a, 130b, and 130c, and a disk 535 at the distal end of the fill material 130, and a center cord 540 that runs the length of the material sections 130a-c. In some embodiments, the cover 120 may be an isolation ring/cover, as described herein. Once delivered into the LAA, the fill material sections 130a, 130b and 130c are configured to absorb blood in the LAA and expand to fill the LAA.

The disk 535 performs two functions:
1) Creates a point to connect the center cord 540 holding the fill material sections 130a-c and cover 120 together; and
2) Creates a fluid barrier to keep blood/water from being absorbed by fill material 130 prior to delivery.

In some of the embodiments, the fill material sections 130a-c may be the same configuration, having the same length and same expanded diameter. In other embodiments, the fill material sections 130a-c may be different configurations, having differed lengths and/or different expanded diameters. In some embodiments, the fill material sections 130a-c are made of the same material with the same material properties. In other embodiments, the fill material sections 130a-c are made of different materials and may have different properties. The properties may include both mechanical properties and electrical properties.

FIG. 27A shows initial delivery of the disk 535 and distal fill material section 130c into the LAA through a delivery device 500. The disk 535 may be configured to create a fluid barrier within the delivery device 500 prior to delivery. Once the fill material 130c is delivered from the distal end 511 of the delivery device 500 into the LAA, the fluid contacts the fill material 130 and it starts to expand. Each subsequent fill material section 130 will also exit the delivery device 500 and absorb blood and expand.

FIG. 27B shows distal fill material section 130c fully expanded, intermediate fill material section 130b expanding, and proximal fill material section 130a starting expansion. After the fill material sections 130a-c are delivered, a cover 120 is then delivered and positioned within the LAA ostium. The delivery device shaft 510 is then disconnected or uncoupled from the cover 120.

FIG. 27C. shows the implant 100 with the multiple fill material sections 130a-c fully expanded in the LAA with the cover 120 positioned within the LAA ostium. The center cord 540 is coupled to the distal side of the cover 120 and the disk 535 and is configured to hold the fill material sections 130a-c and cover 120 together.

In some embodiments, a center connecting cord 540 (center spline) may be used to connect each polymer fill material section 130 and the ostial ring/cover together. This is accomplished by the center connecting cord 540 running through the center of each polymer fill material section 130a-c from the most distal end to the most proximal end. The center connecting cord 540 accomplishes three-functions:
1) connects all the components of the implant 100 together;
2) keeps the delivery device 500 properly centered during implant 100 delivery;
3) allows for variations in LAA anatomy by adjusting the length of the Center Connecting Cord between polymer fill material sections 130a-c so that the implant 100 can accommodate different shapes and sizes of LAA's.

The rate of expansion of the fill material 130 may be adjusted, either faster or slower, to optimize placement in the LAA. A modest rate of expansion on deployment into the distal end of the LAA should provide an opportunity for optimizing axial position during deployment, while the fill material 130 is self-centering, thereby reducing the dependency on precise transseptal puncture position and delivery catheter curvature.

It is possible that a single fill material 130 (e.g. cellulose, open-cell polyurethane, other) will not achieve the requirements to fill the LAA. Therefore, a multi-part, or multi-staged fill material 130 construction may be desirable. For example, a soft foam fill material 130a, (e.g. polyurethane), may engage the LAA tissue (for the reasons stipulated above) with possibly a hollow core for a second-fill material 130b or an inner layer component. This second, inner fill material 130b will be used to generate the needed radial anchoring force by filling the core and pressing on the outer fill material 130a layer against the LAA. The configuration using a separate second-material fill 130b enables a design where the physician can determine the amount of the inner, second fill 130b to be delivered, e.g. deciding when enough material 130 has been placed and stop, trimming off the delivered amount to facilitate an accurate proximal end position of the material fill 130 and then deploy the cover 120.

In some embodiments, the implant 100 may include two fill materials 130a, 130b, the first fill material 130a is configured to fill the LAA to prevent thrombus creation and the second fill material 130b is configured to anchor the implant 100. The first fill material 130a may be any of the materials described herein and the second material 130b may be a spring-like material.

Some fill materials 130 may be thrombogenic by design being made of polyurethanes, which are widely considered hemocompatible. The polyurethane foam may be a porous structure that creates hemodynamics that are favorable for thrombogenesis. When blood passes through the tortuous anatomy of the foam porous structure, the blood experiences low shear stresses and extended residence times that lead to clot formation. These hemodynamics are compounded by the foam's high surface area, which triggers the pathway of the intrinsic clotting cascade that is mediated by contact with foreign material surfaces. After absorbing the blood, platelet adhesion, activation, and fibrin crosslinking is complete in a very short time. For example, between 1 minute and 30 minutes.

One solution to this issue is to use a combination fill material 130 that combines a polymer foam with a blood absorbing polymer. The foam is the core of the implant 100 and the blood absorbing polymer is what fills into the internal surface area of the LAA to provide the mechanical anchoring.

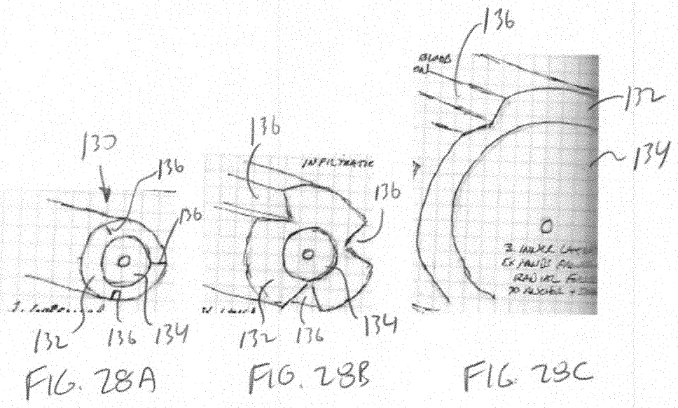

FIGS. 28A-28C show one embodiment of a fill material 130 being made of two materials, a polymer outer foam 132 on an inner foam 134 configuration. The outer foam 132 is a very soft polyurethane foam configured to conform to the surface morphology of the LAA interior, the inner foam 134 is configured to swell with blood to provide the radial force for anchoring/sealing, etc. The outer foam 132 may also include wedge slices 136 that may be needed for blood infiltration. The design should limit the exposure of the inner foam 134 because it could possibly be thrombogenic.

During delivery, the two materials 132, 134 of the fill material 130 are delivered in a compressed configuration, shown in FIG. 28A. FIG. 28B shows the outer foam 132 expands first and allows for repositioning within the LAA, if needed. FIG. 28C shows both the outer foam 132 and inner foam 134 expanded. The inner foam 134 expands and provides a radial force, and the outer foam 132 expands and contacts and fills the peaks and valleys of the LAA anatomy.

In some embodiments, the fill material 130 may be a shape memory polymer (SMP) foam that is formulated to have “memory” properties, allowing it to transition between pre-set shapes. The SMP foam is constructed in its original expanded shape, then compressed into a secondary shape to allow for catheter delivery. Once delivered into the LAA, the SMP foam recovers its original expanded shape.

In some embodiments the fill material 130 is an open cell polyurethane foam or reticulated polyurethane foam. The open cell foam may be used as an alternative to the SMP foam. The clotting cascade with an open cell foam may be shorter than the SMP foam, and the open cell foam may also clot in less than 2 minutes. Reticulated polyurethane foam has been used in blood contact products for decades, such as blood filters and defoaming canisters in the pump loop.

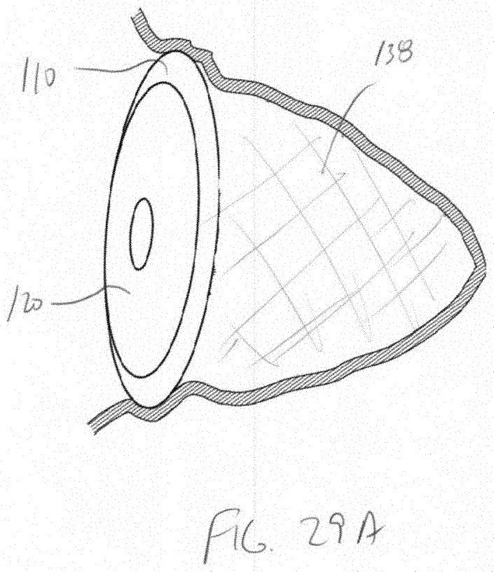

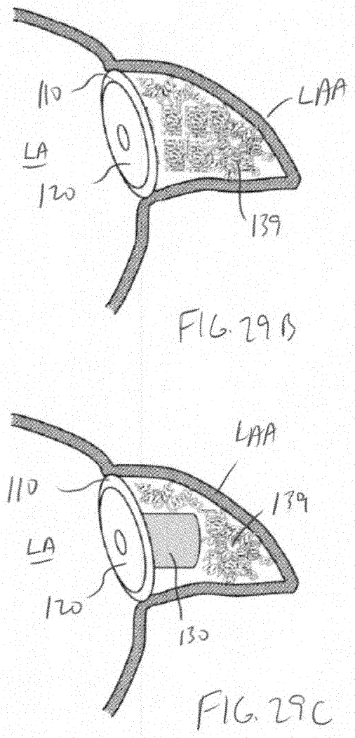

FIG. 29A shows one embodiment of an implant 100 in which the fill material 130 includes a conformable balloon 138 delivered through a catheter-based delivery device 500. There are a number of advantages of using a balloon 138 with a fill material 130. The balloon 138 may be configured to fill the LAA and retain the fill material 130 to assure it cannot escape past the ostium during filling. And based upon the fill material 130 properties, a balloon 138 may allow for withdrawal, repositioning, or full retraction for bail-out before being “set”. This configuration may minimize catheter delivery and deployment issues, while potentially achieving good radial force after deployment. This embodiment may also provide for repositioning within the LAA if needed prior to completing the fill.

In one embodiment, the balloon 138 is supple enough to conform to the complex interior of the LAA for complete isolation when filled. In another embodiment, the balloon 138 may only be required to conform and anchor at the “neck” near the ostium for sufficient isolation.

In some embodiments, the implant 100 includes an entirely inflatable system with an inflatable balloon 138 filled with an in-situ polymerizing fluid fill material 130. The polymerizing fluid fill material 130 may be a liquid plastic polymer that hardens once the balloon 138 fits into place.

In some embodiments, the fill material 130 may be a photocure polymer that is cured with a UV light source (fiber or LED). In this embodiment, a liquid fill material 130 is delivered to the LAA and positioned within the LAA in the proper location. Once in position, a light source is used to cure the fill material 130 in place, turning the liquid material into a solid material within the LAA. By using a liquid fill material 130, the physician uses his discretion on the amount of liquid fill material 130 to use, eliminating the need to preoperatively measure the volume of each LAA (they vary widely across patients), and pre-selects a foam fill size that is to fit that volume (or shape), as discussed above.

In some embodiments, the implant 100 includes an elastomeric/compliant balloon 138 attached to a flexible fill tube designed for polymer fill. The attachment of the balloon 138 to the tube should be leak free and secure. The balloon 138 and fill-tube reside within (inside) an intermediate sized PTFE flexible tube, e.g. a soft “delivery-cartridge tube” that holds the balloon 138 and fill tube and allows for protection of the balloon. The “delivery-cartridge tube” is configured to fit inside a delivery sheath. As such, there is no physician direct contact with the balloon 138 or small/thin fill tube.

During use, the delivery sheath is positioned over a pigtail catheter at the LAA ostium but is positioned to stay out of the LAA. The delivery sheath is placed at the LAA ostium just deep enough to avoid loss of access/falling-out of ostium into atrium. The pigtail is then removed. The delivery-cartridge tube is inserted into the proximal end of the sheath and advanced down the lumen to the LAA like it was a guidewire. This PTFE delivery-cartridge tube is soft and is advanced past the distal end of the sheath and enters the LAA. This delivery-cartridge tube (with the balloon 138 and fill tube inside) may come into contact with the LAA and is less likely to cause tamponade/perforation than a thick, reinforced sheath. There are marker bands at the distal end of the cartridge tube to identify the position under fluoro. The distal end of the delivery-cartridge tube is placed distal in the LAA under fluoro, regardless of LAA shape (e.g. broccoli type, chicken wing type, wind sock type). Contrast can be injected through the sheath, around the delivery-cartridge tube.

With the fill tube held in position at the proximal end, the delivery-cartridge tube is retracted to expose the balloon 138 and continues until the distal end of the fill tube is also exposed. The sheath is not moved.

A fill material 130 is injected down the fill tube into the balloon 138. In some embodiments, the fill material 130 is a liquid, in-situ polymerizing agent. This agent may have some amount of RO filler so that the filling of the balloon 138 can be observed and adjusted to position the delivery cartridge or fill tube as needed to get the balloon 138 to conform to all of the major LAA features. As the balloon 138 fills, the focus is to assure filling in the distal lobes for an anchor and complete filling around the “neck” (the LAA's shape approaching the ostium) for a seal. The fill tube and cartridge tube can each be manipulated axially (e.g. in and out) as needed to assure this neck portion of the balloon 138 is filling completely. The liquid polymer agent stays under modest injection pressure. The system allows some forgiveness on proper fill volume, e.g. the LAA may distend slightly without injury or concern, and it may be slightly underfilled in the distal lobes if it is at least sealed around the neck. Contrast injections from the sheath may be done during fill to help discern proper filling amount (when to stop filling).

Before cure, the fill tube can be used for a light tug test to assure that the filled balloon 138 is anchored well in the LAA.

Once filled, if the polymerizing agent is photocure polymer, for example Mosaic, UV light is applied, for example, UV light trans-illuminated down the wall of the fill tube. The light starts the cure process, but the polymer is assured of complete cure even without complete illumination. The agent cures in the balloon 138 and within the fill tube in seconds. Nothing can escape in a liquid form after this step.

A cover, such as a self-expanding NiTi structure, is delivered, passed down the sheath, over the fill tube. As the cover reaches the end of the sheath, it expands to the correct diameter in the ostium, it remains centered on the fill tube and centered in the ostium shape. As the cover reaches the fill tube/balloon 138 junction, a deployment system causes the balloon 138 to detach from the fill tube and connect to the cover 120, anchoring the cover 120 to the polymer filled balloon 138.

In some embodiments, the cover 120 may contain a ring for acute lesion and tissue compression to isolate the LAA, such as described herein.

FIG. 29B shows one embodiment using a fill material 130 that includes “coils” 139 inserted into the LAA, similar to coils used in neurovascular aneurysm repair. The coils 139 may be delivered using a catheter system. Once the delivery system is in position, the coils 139 are pushed out of a catheter into the LAA and then conform to the LAA. The coils 139 make a random mechanical mass with some radial force as the mass builds, thereby providing flexibility in these filling applications. The coils 139 can be provided in different lengths and may be deployed sequentially until the physician believes the LAA space is adequately filled based upon the end goals established. Before the coils 139 are released (e.g. if they are “detachable coils”), they can be pulled back or withdrawn entirely. The coils 139 are solid and/or not capable of expansion, which is both limiting for filling spaces but also beneficial in deploying through a flexible catheter.

In some embodiments the coils 139 are platinum coils attached to a catheter for delivery. When the catheter has reached the LAA, an electrical current may be used to separate the coils 139 from the catheter. The coils 139 are left in place permanently in the LAA. Depending on the size of the LAA, multiple coils 139 may be needed to completely fill the LAA.

The coils 139 may be made of soft platinum metal and may be different shapes or sizes, shaped like a spring. The coils 139 are very small and thin, ranging in size from about twice the width of a human hair (largest) to less than one hair's width (smallest).

FIG. 29C shows an embodiment in which the coils 139 are used with fill material 130.

In some embodiments, the fill material 130 may be a shape memory polymer (SMP) foam. The SMP foam may be a porous polymeric foam that is formulated to have “memory” properties, allowing it to transition between pre-set shapes at different temperatures and aqueous environments. The shape memory capacity of these foams results in an ideal material for minimally invasive devices which provide limited friction during catheter delivery. However, they are still capable of expanding up to ten times their crimped diameter to fill large volumes and create rapid occlusion of vessels with a single device. The affinity for rapid clot formation is primarily due to the high surface area and porous morphology of the foam that creates numerous recirculation and stagnation zones that activate rapid thrombosis.

The SMP foam is constructed in its original expanded shape, then compressed into a secondary shape to allow for catheter delivery. Once delivered into the LAA, the SMP foam recovers its original expanded shape.

In some embodiments, system 10 is configured to stimulate the heart of the patient. In these embodiments, the implant 100 may include a sensor 150 and a second implant 300 that includes a stimulation device, such as stimulating electrodes incorporated therein or on the surface, adapted to be in contact with heart tissue. For example, if an arrhythmia is detected with the sensor 150, the stimulation device can be adapted to pace cardiac tissue through electrical stimulation thereof. In some embodiments, the second implant 300 can be adapted to deliver a therapeutic compound to the patient in the event an arrhythmia is detected.

The sensor 150 of the first implant 100 is configured to record electrical activity of the heart, and the stimulation device of the second implant 300 is configured to pace the patient's heart when the system detects an abnormal cardiac rhythm based on the recorded electrical activity. System 10 can be configured to deliver the stimulation in a closed loop fashion, such as based on physiologic parameters recorded by sensor 150 of first implant 100 or other sensors of system 10. Sensor 150 can be configured to record ECG of the patient, and the second implant 300 can deliver stimulation energy to pace the heart. For example, system 10 can be configured to detect atrial fibrillation (AF) and, when detected, cause first implant 100 and/or second implant 300 to pace the heart to cardiovert the patient. Alternatively or additionally, system 10 can be configured to detect lack of a pulse, ventricular fibrillation, (VF), and/or pulseless ventricular tachycardia (VT), and, when any are detected, cause first implant 100 and/or second implant 300 to defibrillate the heart.

Second implant 300 can be implanted in a surgical procedure or via one or more catheter-based delivery devices in an interventional percutaneous procedure (e.g. an over-the-wire interventional procedure). In some embodiments, second implant 300 is positioned proximate the heart of the patient, such as when second implant 300 is configured to pace and/or defibrillate the patient's heart.

Second implant 300 can comprise an electronics module 170, comprising various components configured to collect and/or process information, store information, transmit information, receive energy and/or transmit energy (e.g. to one or more electrodes or other energy delivery components of second implant as described herein). Electronics module 170 can comprise an energy storage element, power supply 171 (e.g. a battery, a rechargeable battery, and/or a capacitor). Electronics module 170 can comprise memory circuitry, memory 172, configured to store information. Electronics module 170 can comprise a microcontroller or data processing assembly, processing unit 173. Electronics module 170 can comprise one or more algorithms, algorithm 174, such as an algorithm configured to analyze data recorded by second implant 300. Electronics module 170 can comprise a module configured to transmit data (e.g. to a separate device within or outside of the patient), transmitter 175. In some embodiments, transmitter 175 is configured to receive data, such as when transmitter 175 transmits and/or receives data to and/or from communication device 200 and/or implant 100. Transmitter 175 can comprise one or more antennas configured to transmit and/or receive data (e.g. to and/or from implant 100 and/or communication device 200), and/or one or more antennas configured to receive energy (e.g. from communication device 200 and/or implant 100).

As described hereabove, in some embodiments second implant 300 transfers information to and/or from first implant 100. Alternatively or additionally, energy can be transferred between second implant 300 and first implant 100, such as via inductive or other wireless energy transfer.

Delivery device 500 can comprise one or more catheters 510 or other tools configured to deliver a separate device, such as to deliver implant 100. In some embodiments, delivery device 500 is configured to deliver energy to implant 100. In some embodiments, delivery device 500 is also configured to deliver fill material 130 to implant 100 (e.g. after the other components of implant 100 have been positioned in the LAA).

Delivery device 500 is constructed and arranged for insertion into a body location, such as the pulmonary vein. Delivery device 500 includes a shaft 510 having a distal end 511 and proximal end constructed of sufficiently flexible material to allow insertion through the tortuosity imposed by the patient's vascular system. The shaft includes a lumen traveling from the proximal end to the distal end 511. The lumen is constructed and arranged to allow the implant 100 to be slidingly received by lumen. The implant may be positioned within lumen during insertion of the delivery catheter 500 or may by inserted through the lumen after delivery catheter 500 is positioned within the LAA.

In some embodiments, the implant 100 is pre-loaded into the distal end 511 of a delivery device 500 or thin-walled sleeve, such as to be completely isolated from fluid to minimize the potential for fluid (e.g. saline or blood) uptake/ingress into the implant 100 before being delivered into the LAA. Once the sheath is properly placed, the pre-loaded delivery device 500 is advanced through the sheath to the distal end 511. Radiopaque markers on delivery device 500 help physicians guide the catheter's placement, the pre-loaded implant delivery device 500 is advanced out the distal end 511 of the sheath and into the distal portion of the LAA, exposing the implant 100 material to the blood for expansion.

In some embodiments, a shaft or shaft-like structure is used to advance the implant 100 and deliver from the sleeve 520. The pusher will allow the physician to align the angle of the cover 120 to the LAA Ostium. The shaft may then be detached from the implant 100.

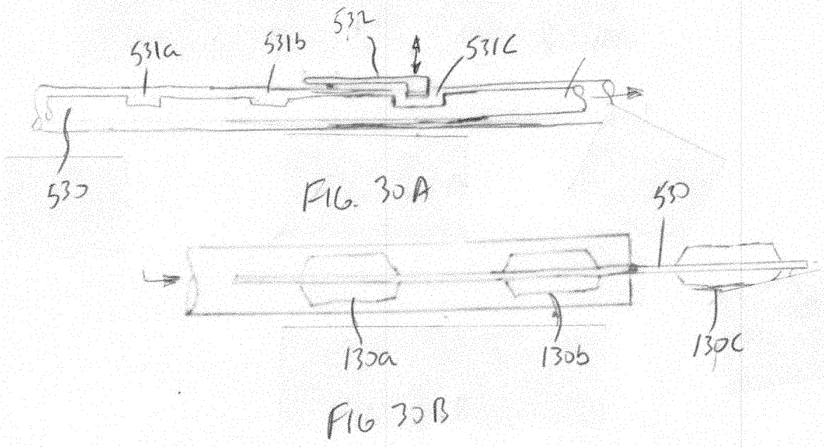

FIG. 30A is a proximal sectional view of the delivery device 500 near the handle or part of the handle of the catheter and FIG. 30B is the corresponding distal end of the delivery device 500. In the embodiment shown, three fill material 130a-c and/or implant 100 in a compressed configuration are mounted on a delivery rod 530 positioned for delivery within a lumen of the delivery device 500. The proximal portion of the delivery rod 530 includes notches 531a-c in the delivery rod 530 that correspond to a position between each fill material 130a-c on the rod 530. A lever 532 is configured to engage each notch 531a-c and stop movement of the delivery rod 530. The lever 532 may be spring biased in the closed position and must be raised to release the notch 531a-c. The lever 532 can then be raised and the rod 530 may be advanced to the next notch 531a-c. The notches 531a-c identify when the fill material section 130a-c has been fully deployed. In FIG. 30B, fill material 130c has been delivered from the end of the delivery device 500. Once an appropriate amount of time has passed to allow the fill material section 130a-c to absorb the blood and expand, the lever 532 is moved and the rod 530 is advanced until the lever 532 engages the next notch 531a-c to indicate that the next fill material section 130a-c has been delivered.

In some embodiments, the delivery device may first deliver a ring 110, followed by a fill material 130 and then a cover 120. The ring 110 may be similar to the isolation rings discussed herein to compress the tissue for isolation of the LA and LAA. The fill material 130 is then deployed, such as the blood absorbing fill material 130, or other fill materials 130 described herein. Once delivery of the fill material 130 is complete, a cover 120 is deployed and positioned at the ostium. The ring 110 may be deployed at the LAA ostium, or it may be designed to also work in other parts of the body, such as the pulmonary vein.

FIGS. 31A and 31B show one option for delivery of the implant 100 in a delivery device 500 using a guidewire 530, J-tip guidewire 530. Other tips may also be used, such as straight tip or angled tip. FIG. 31A shows the distal end of the delivery device 500 positioned proximate the LAA. The guidewire 530 is then extended from the catheter into the LAA. Once in the LAA, the fill material 130a-c may be delivered, shown in FIG. 31B. After delivery of the fill material 130, the ring 110 and cover 120 are positioned in place.

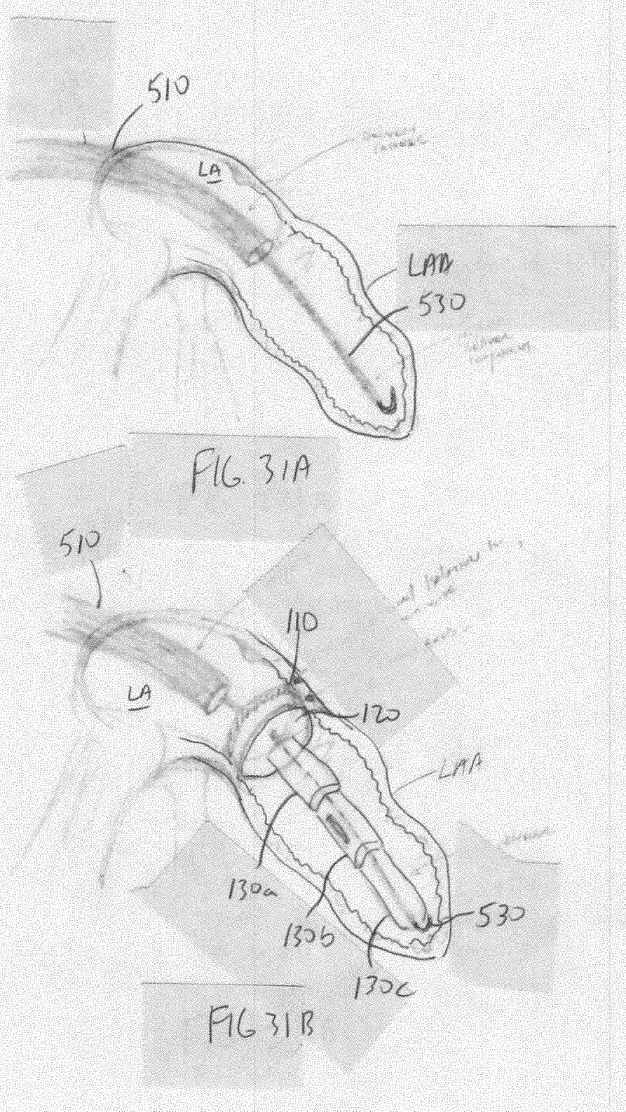

FIG. 32 shows one embodiment of a delivery device 500 having a transseptal sheath 542 with an elongate shaft 510 with a distal portion 544. Mounted to distal portion 544 is an expandable LAA occlusion balloon 546, which is configured to be inserted into the ostium of the LAA and expanded to seal the OS. The transseptal sheath 542 incorporates an occlusion balloon 546 for closing the LAA and preventing blood flow to the site of the perforation.

In some embodiments, the LAA occlusion balloon 546 is used to seal the LAA after a perforation. The LAA being fragile, is prone to perforations, and if a perforation is created, patient safety requires acute attention. Traditionally, a surgical team is called to the patient to open the chest cavity and repair the LAA perforation. The use of a transseptal sheath 542 that incorporates an occlusion balloon 546 for closing the LAA and preventing blood flow to the site of the perforation provides a safe option to a surgical approach in treating the LAA perforation.

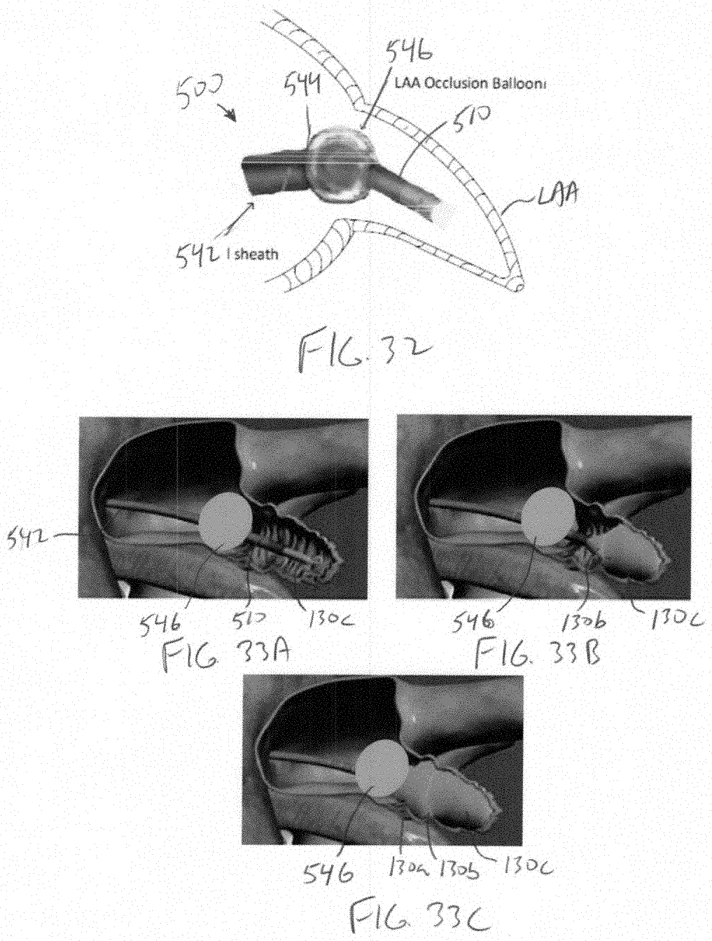

FIGS. 33A-33C show one embodiment of the LAA occlusion balloon 546 used with a delivery device 500 delivering a fill material 130, as described herein. Once the LAA occlusion balloon 546 is inflated at the ostium, the delivery device 500 delivers one or more fill materials 130, such as 130a-c. The balloon 546 may be used as a centering tool to position the delivery device 500 at a center of the ostium for delivery of the implant 100 to the LAA. This may prevent perforations of the LAA. If there are perforations present, fill material 130 will absorb and expand with the blood in the LAA and may close and/or seal off any perforations of the LAA that may have occurred.

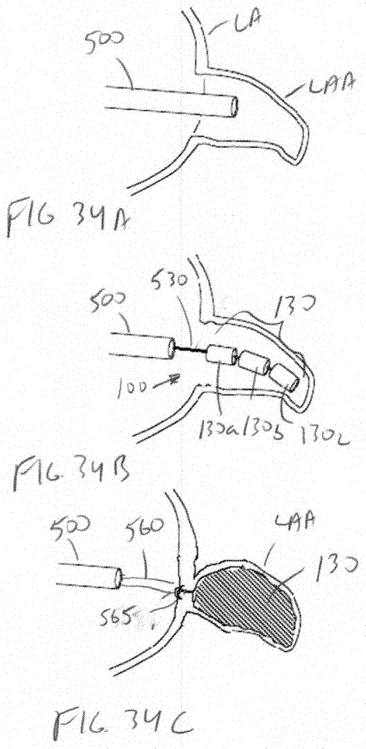

In some embodiments, it may be desirable to stitch the LAA closed after placement of the implant 100. Referring now to FIGS. 34A-34C, a series of steps that fluidly isolates the left atrial appendage is illustrated, including stitching the LAA closed. FIG. 34A, shows the delivery device 500 advanced into the LA toward the LAA. FIG. 34B shows implant 100 being implanted, with the fill material 130 being deployed within the LAA. In the embodiment shown, fill material 130 includes multiple fill material sections 130a-c that are coupled together. In addition to the fill material 130, other elements of the implant 100 may be delivered to the LAA, such as sensors 150, electronics 170 (LA PSI and ECG loop-recording), etc.

FIG. 34C shows the fill material 130 expanded within the LAA. A stitching device 560, either a stand-alone device or a device inserted through the delivery device 500, is advanced and configured to engage the LAA tissue near the ostium and a stitch 565 is performed to close the LAA. The stitching device 560 may be similar to the Nobel Stitch sold by HeartStitch. Other types of closure methods may also be used, such as: tissue welding, adhesive, staples or other suitable closure methods. Once the LAA is closed, the stitching device 560 may be detached and device 560, 500, is withdrawn.

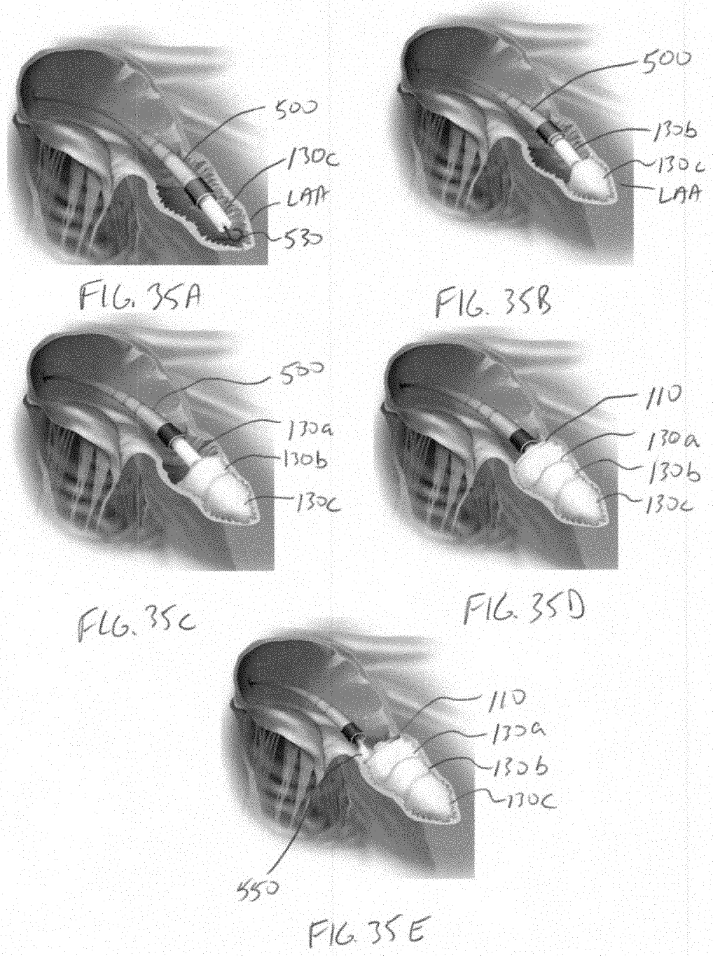

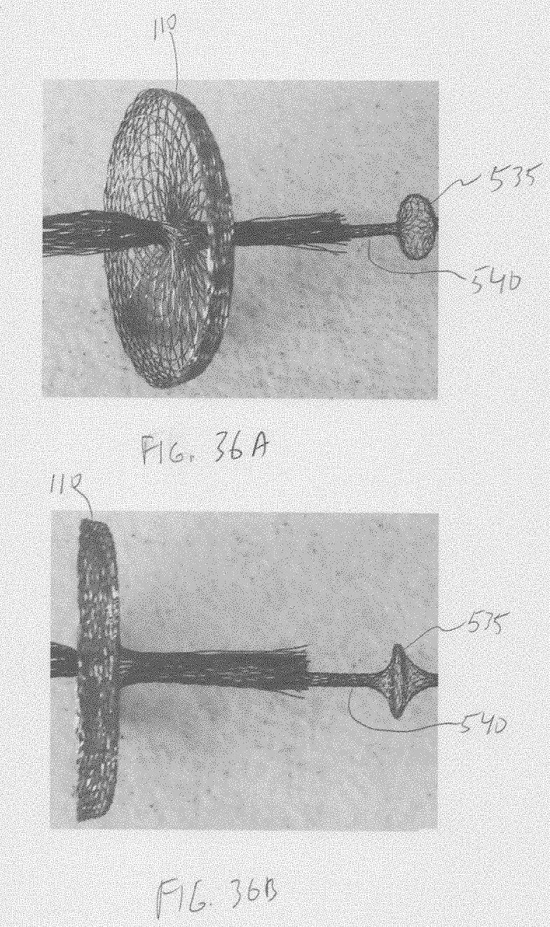

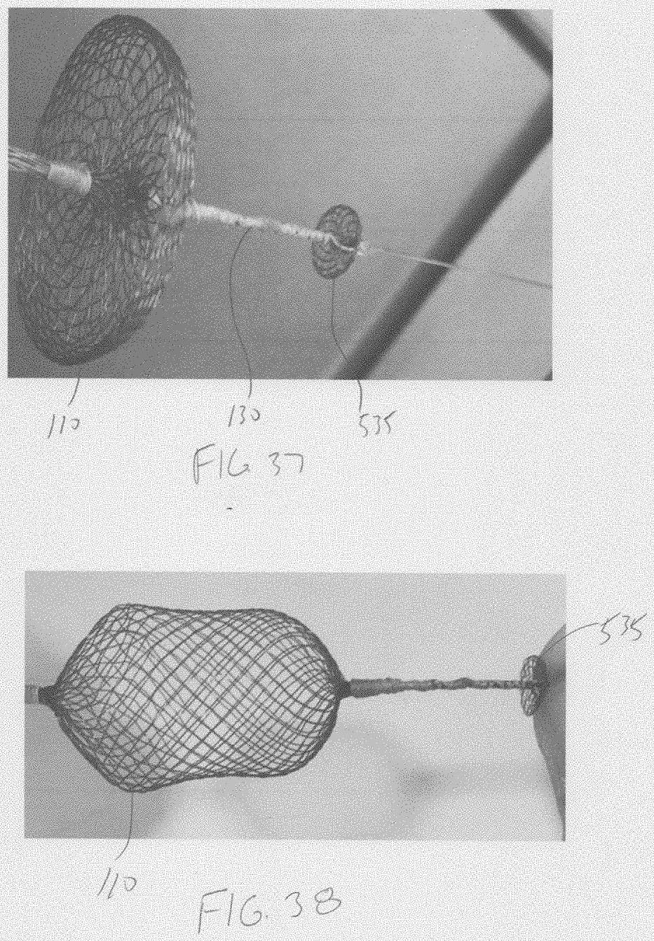

FIGS. 35A-35E show one embodiment for deployment of a blood expanding fill material 130 in the LAA. In the embodiments shown, the LAA is filled with fill material 130 from the distal end first. This ensures that the fill material 130 is properly deployed and anchored at the most distal end of the LAA.

Step 1: The first, distal material section 130c is deployed by positioning the delivery device 500 near the distal end of the LAA. Slowly push the first fill material section 130c out the distal end of the delivery device 500, while also slightly pulling delivery device 500 proximally. This allows the first fill material section 130c to fully expand into the LAA distal end by absorbing the blood and filling the space previously occupied by the blood and generating a mechanical locking with the inside of the LAA's anatomy (i.e. trabeculations, peaks and valleys).

If the first fill material section 130c is not properly deployed within the LAA, removal is still possible.

Step 2: Once Step 1 is complete, delivery device 500 is self-centered via the fill material sections 130a-c center connecting cord 530.

The deployment of the second fill material section 130b is initiated. The delivery mechanism is held in place while the delivery sheath 542 is slightly pulled back. As the sheath 542 is pulled back, the second fill material section 130b becomes exposed to blood and will start absorbing the blood and expanding.

Continue pulling back the delivery sheath 542 until the second fill material section 130b is completely outside the sheath.

Step 3: Once step 2 is complete, the deployment of the third fill material section 130c is initiated.

The delivery mechanism 500 is held in place while the delivery sheath 542 is slightly pulled back. As the sheath 542 is pulled back, the third fill material section 130c becomes exposed to blood and will start absorbing the blood and expanding.

Continue pulling back delivery sheath 542 until the third fill material section 130a is completely outside the sheath 542.

Ensure that no fill material 130 is outside the ostium of the LAA. If portions protrude into the LA, use the delivery sheath 542 to push material into the LAA.

Step 4: The distal end 544 of transseptal sheath 542 should be located near the ostium of the LAA.

Deploy LAA ostial ring 110 by advancing the ring 110 out of the sheath 542.

Fine tuning the position can be accomplished by pushing and/or pulling the sheath 542 while it remains attached to the delivery mechanism 500. Final implant 100 verification can be performed via fluoroscopy.

Once final position is verified and satisfactory, release implant 100.

Step 5 (optional): The physician may choose to use an ablation catheter 500 to deliver energy 550 through the ostial ring 110 into the LAA ostial tissue. In this embodiment, the ostial ring 110 is a conductive ring, as described herein.

FIGS. 36A and 36B show one embodiment of an implant having a single wire-form/braid with a proximal wire mesh LAA ostial ring 110 and a distal wire mesh disk 535 connected by a connective wire mesh cord 540. The wire mesh cord 540 may be configured to:
1) connect all the components of the implant 100 together;
2) keep the delivery device 500 properly centered during implant 100 delivery;
3) allow for variations in LAA anatomy by adjusting the length of the Center Connecting Cord between polymer fill material 130 so that the implant 100 can accommodate different shapes and sizes of LAA's.

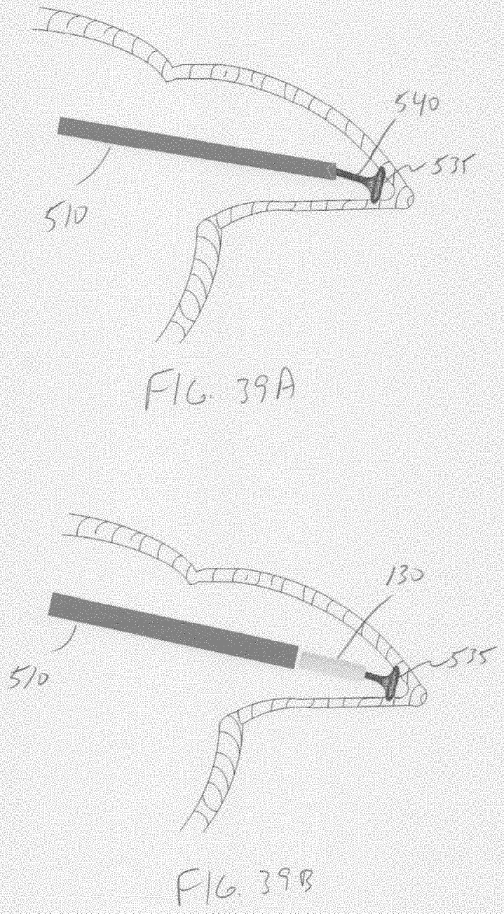

FIG. 37 shows the polymer fill material 130 wrapped around the center cord 540 connecting the distal wire-disk 535 to the larger, proximal wire-form 110. The polymer material is configured to absorb water from the blood and expands, filling the space. In some embodiments, the fill material 130 may be a shape memory polymer (SMP) foam that is formulated to have “memory” properties, allowing it to transition between pre-set shapes. The SMP foam is constructed in its original expanded shape, then compressed into a secondary shape to allow for catheter delivery. Once delivered into the LAA, the SMP foam recovers its original expanded shape.

The device may also include a cover 120 coupled to the wire mesh ring 110. The LAA Ostium ring & cover may be made of a nitinol wire mesh (or similar metal) that conforms to the shape of any LAA and is configured to apply a uniform outward force in all directions to fully expand into the ostium of the LAA.

FIG. 38 shows the device being elongated and then compressed for delivery. After delivery, the device is allowed to return to its set state for deployment in the LAA.

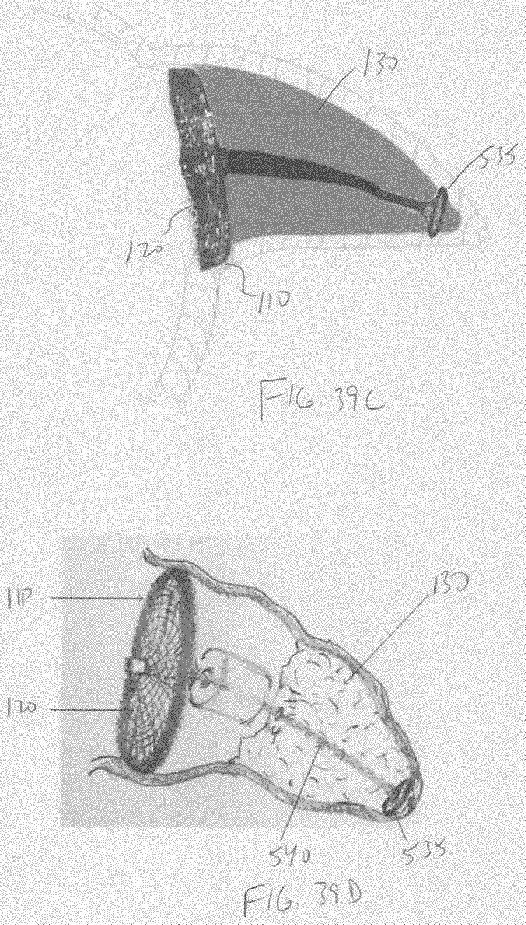

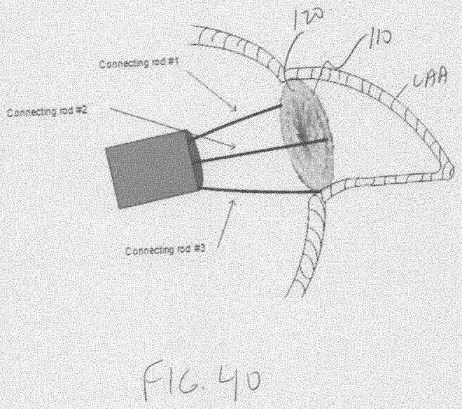

FIGS. 39A-39D show deployment of the compressed device from the end of a catheter or sheath 510.

Step 1; Place the sheath 510 in the distal end of the LAA the pull back the sheath 510 to deliver the distal wire-form anchor segment 535 into the distal end of the LAA

Step 2; Pull back the sheath 510, exposing the polymer 130 to the fluid in the LAA. In some embodiments, the polymer is a PET fiber with impregnated hydrogel and the hydrogel absorbs the water/fluid in the blood and expands, the fiber material keeps the polymer held together as one cohesive component, which is connected to the center-cord of the nitinol wire braids 540.

Step 3; After polymers 130 is delivered and expanded into LAA, deliver the Large Nitinol braid (wire-form) 110 with a polyester fabric or cover 120 coupled to the braided disk 110.

FIG. 40 shows one embodiment of a device designed to provide a 3-point position concept. Braided structure 110 is deployed in LAA and “wedged” into LAA ostium with the 3-point alignment system for fluid isolation. The alignment system could be tubes for injecting fluids or delivering energy, then disconnected. Space exists to add monitoring electronics in the implant body. Potential exists to add RF electrode(s) along the implant perimeter.

In some embodiments, the device is designed to provide LAA acute isolation using irreversible electroporation. In the configuration shown, acute LAA electrical isolation can be achieved by Irreversible Electroporation (IRE); high voltage (˜2000V/cm), ultra-short electrical pulses (25-75 μS) applied to LAA Ostium ring 110 of the device. The three (3) positioning or connecting rods #1, #2, #3 will fine tune the ostial ring/cover, once the positioning is completed. The proximal end is connected to an RF generator to deliver RF ablation energy to LAA ostium, ensuring acute electrical isolation. Long-term electrical isolation occurs from the “pressure mediated” approach, where the constant outward pressure on the LAA ostium compresses the myositis, preventing the normal ion exchange, rendering the cells inactive, and over-time the cells die and form fibrosis. This achieves long-term/permanent lesion creation (scar tissue).

The embodiment may also include an Irreversible Electroporation (IRE) Ablation Generator that is:
1) Battery powered,
2) Output: Irreversible Electroporation (IRE); IRE uses electrical energy to target cardiac tissue at the cellular level
   2a) Ultra-short duration electrical pulses,
   2b) Field strength: 2000 volts/cm; approx. 50 A (tissue- and geometry dependent)
3) temperature monitoring
4) Impedance monitoring
5) Capable to output cardiac pacing signals to test and verify electrical isolation
6) Cycle the ultra-short electrical pulses between rods #1 and #2, #2 and #3, #3 and #1 for complete circumferential ablation

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

What is claimed is:

1. A system for treating a medical condition of a patient, comprising:
an implant configured to be inserted into a left atrial appendage (LAA) of the patient,
wherein the implant comprises a multi-component design, an ostium ring with ostial cover configured to engage the ostium of the left atrium LAA to separate the LAA from the left atrium (LA) and fill material configured to engage LAA.

2. The system of claim 1, wherein the implant is configured to fluidly isolate the LAA.

3. The system according to claim 2, wherein to fluidly isolate the LAA from the heart and prevents clot/thrombus formation inside the LAA from exiting the LAA.

4. The system according to claim 1, wherein the ostium ring is self-expanding.

5. The system according to claim 1, wherein the ostium ring provides fluid isolation of the LAA.

6. The system according to claim 1, wherein the fill material comprises multiple fill portions.

7. The system according to claim 1 wherein the fill material is configured to anchor the implant.

8. The system according to claim 1, wherein the fill material comprises shape memory material.

9. The system according to claim 1, wherein the ostium ring provides equal pressure around a non-circular shaped ostium.

10. The system according to claim 1, wherein the implant includes a combination of LAA occlusion with electrical isolation.

11. The system according to claim 1, wherein the implant includes a multi-part implant that enables precise positioning.

12. A system for treating a medical condition of a patient, comprising:
an implant configured to be inserted into a left atrial appendage (LAA) of the patient,
wherein the implant comprises a multi-component design including:
an ostium ring with ostial cover configured to engage the ostium of the left atrium LAA and fluidly isolate the LAA from the left atrium (LA), and
a fill material configured to engage LAA.

13. The system according to claim 12, wherein the ostium ring is self-expanding.

14. The system according to claim 12 wherein the fill material is configured to anchor the implant.

15. The system according to claim 12, wherein the fill material comprises shape memory material.

16. The system according to claim 12, wherein the ostium ring provides equal pressure around a non-circular shaped ostium.

17. The system according to claim 1, wherein the implant includes a multi-part implant that enables precise positioning.

18. A system for treating a medical condition of a patient, comprising:
an implant configured to be inserted into a left atrial appendage (LAA) of the patient,
wherein the implant comprises a multi-component design including:
a self-expanding ostium ring with ostial cover configured to engage the ostium of the left atrium LAA and fluidly isolate the LAA from the left atrium (LA), and
a multi-part fill material configured to engage LAA. and anchor the implant.

19. The system according to claim 12, wherein the multi-part fill material comprises shape memory material.

20. The system according to claim 1, wherein the implant includes a multi-part fill material that enables precise positioning.
